SEQUENCE LISTING (17 sequences)

SEQ ID NO: 1 - DNA, 3677 bp, Stachybotrys chartarum
ctggctagcctcacttggtagacagccctgacagcctcactggctgggggtcgaaaggccagtcaatatcttggtcactgctaatagttccttgctacgcgcaaaaagctccttgccgaaggggcacagactatcaagtgagacatataggatgcatgtctttcatagccacagttagggtggtgacctactcgaagaggccccgacttgcatgcatacgacatgtcgcttccatgcaacatgtatgcgcacatcggcgatcaggcaccctctgcatgcagaatagaacccccctggtttccttttgtttcttttcctttctcaacgacgcgtgagcgtggttaacttgagcaaggccgagtggtctgttcacgaggttaccatcgaactctcttctttcccaatcatgacctgccccccgagtttagcccccatcacggctgtgaaatccacttcgataatcctagcctagtgctactcttcaatagttgctcctgatggggcactttggtcacattgccttggttyctcctacctcgttctcttccgcatcaagcctctatgcccgacgacaacacctcattggcccggaccactttgagcgcgcacgcaccttcgcgccgaaggagttgataacacccttcacccttgcccaatgatggagttttggtctatttgtcatgatcacctcacattcactagatcacggatcctggaagagggtgtggaagccagaccagcttgtccctgttcttgcagactcaggtcagctcctagcggctatcacagctcaggattatcaagtcccgtaaagtccagacccttttcattgtatgatgctgcctaatttgcgctatctctatgccgtagcagccgtcttggctacaactggctgccatggctgaagcatcgtgagatctataaaggtctccgaatcctcggtgaagtcagaatcgtctctccacaccagtcaacaacaagcttctttctcttacagcttagcctgagcacattcacagaactcttcccttcttttcgtcaatatgctgttcaagtcatggcaactggcagcagcctccgggctcctgtctggagtcctcggcatcccgatggacaccggcagccaccccattgaggctgttgatcccgaagtgaagactgaggtcttcgctgactccctccttgctgcagcaggcgatgacgactgggagtcacctccatacaacttgctttacaggtgagacacctgtcccacctgttttccctcgataactaactcttataggaatgccctgccaattccacctgtcaagcagcccaagatgtatgtctttgattttctacgaagcaactcggccccgactaatgtattctaggatcattaccaaccctgtcaccggcaaggacatttggtactatgagatcgagatcaagccatttcagcaaagggtgagtttgctcagaaaccttgtggtaattaatcattgttactgaccctttcagatttaccccaccttgcgccctgccactctcgtcggctacgatggcatgagccctggtcctactttcaatgttcccagaggaacagagactgtagttaggttcatcaacaatgccaccgtggagaactcggtccatctgcacggctccccatcgcgtgcccctttcgatggttgggctgaagatgtgaccttccctggcgagtacaaggattactactttcccaactaccaatccgcccgccttctgtggtaccatgaccacgctttcatgaaggtatgctacgagcctttatctttcttggctacctttggctaaccaacttcctttcgtagactgctgagaatgcctactttggtcaggctggcgcctacattatcaacgacgaggctgaggatgctctcggtcttcctagtggctatggcgagttcgatatccctctgatcctgacggccaagtactataacgccgatggtaccctgcgttcgaccgagggtgaggaccaggacctgtggggagatgtcatccatgtcaacggacagccatggcctttccttaacgtccagccccgcaagtaccgtttccgattcctcaacgctgccgtgtctcgtgcttggctcctctacctcgtcaggaccagctctcccaacgtcagaattcctttccaagtcattgcctctgatgctggtctccttcaagcccccgttcagacctctaacctctaccttgctgttgccgagcgttacgagatcattattggtatgccctcccctctcacgaatgagtcaagaactctaagactaacacttgtagacttcaccaactttgctggccagactcttgacctgcgcaacgttgctgagaccaacgatgtcggcgacgaggatgagtacgctcgcactctcgaggtgatgcgcttcgtcgtcagctctggcactgttgaggacaacagccaggtcccctccactctccgtgacgttcctttccctcctcacaaggaaggccccgccgacaagcacttcaagtttgaacgcagcaacggacactacctgatcaacgatgttggctttgccgatgtcaatgagcgtgtcctggccaagcccgagctcggcaccgttgaggtctgggagctcgagaactcctctggaggctggagccaccccgtccacattcaccttgttgacttcaagatcctcaagcgaactggtggtcgtggccaggtcatgccctacgagtctgctggtcttaaggatgtcgtctggttgggcaggggtgagaccctgaccatcgaggcccactaccaaccctggactggagcttacatgtggcactgtcacaacctcattcacgaggataacgacatgatggctgtattcaacgtcaccgccatggaggagaagggatatcttcaggaggacttcgaggaccccatgaaccccaagtggcgcgccgttccttacaaccgcaacgacttccatgctcgcgctggaaacttctccgccgagtccatcactgcccgagtgcaggagctggccgagcaggagccgtacaaccgcctcgatgagatcctggaggatcttggaatcgaggagtaaaccccgagccacaagctctacaatcgttttgagtcttaagacgaggctcttggtgcgtattcttttcttccctacggggaactccgctgtccactgcgatgtgaaggaccatcacaaagcaacgtatatattggactcaccactgtcattaccgcccacttgtacctattcgattcttgttcaaacttttctagtgcgagagtgtccatagtcaagaaacgcccatagggctatcgtctaaactgaactattgtgtggtctgtgacgtggagtagatgtcaattgtgatgagacacagtaaatacggtatatcttttcctaggactacaggatcagtttctcatgagattacatccgtctaatgtttgtccatgagagtctagctaaggttgagaatgcatcagacggaatcatttgatgctctcagctcgtattaccgatgtaagacaagttaggtaagttgcttggtatccgaaaatgactcaggctccctcattaggttgcatgtgaaaaccttcagcaactcatgggtgttgggaccaaatcatccatacctgattttgataactgacctgggtcaat

SEQ ID NO: 2 - protein, 594 aa, Stachybotrys chartarum
MLFKSWQLAAASGLLSGVLGIPMDTGSHPIEAVDPEVKTEVFADSLLAAAGDDDWESPPYNLLYRNALPIPPVKQPKMIITNPVTGKDIWYYEIEIKPFQQRIYPTLRPATLVGYDGMSPGPTFNVPRGTETVVRFINNATVENSVHLHGSPSRAPFDGWAEDVTFPGEYKDYYFPNYQSARLLWYHDHAFMKTAENAYFGQAGAYIINDEAEDALGLPSGYGEFDIPLILTAKYYNADGTLRSTEGEDQDLWGDVIHVNGQPWPFLNVQPRKYRFRFLNAAVSRAWLLYLVRTSSPNVRIPFQVIASDAGLLQAPVQTSNLYLAVAERYEIIIDFTNFAGQTLDLRNVAETNDVGDEDEYARTLEVMRFVVSSGTVEDNSQVPSTLRDVPFPPHKEGPADKHFKFERSNGHYLINDVGFADVNERVLAKPELGTVEVWELENSSGGWSHPVHIHLVDFKILKRTGGRGQVMPYESAGLKDVVWLGRGETLTIEAHYQPWTGAYMWHCHNLIHEDNDMMAVFNVTAMEEKGYLQEDFEDPMNPKWRAVPYNRNDFHARAGNFSAESITARVQELAEQEPYNRLDEILEDLGIEE

SEQ ID NO: 3 - DNA, 2905 bp, Bipolaris spicifera
gtggcgtcggggatccacctgaatcatgagatataaagagagggatgttctgtcaacaataatcccatcatcagcttttgaacattctcagctcatcaaagattttcttcaagatggtcgccaaatacctcttctcagcacttcaactcgtttcaattgcgaaaggcatatacggygtcgctttgagcgaacgtcccgccaaatttgtcgacaacacccccgacgaagaaaaggctgccttggcgtcaattgttgaagatgaccctgcggatgttgtcaacatgctgaaagactggcaaagcccggagtatcctctcatttttcgccaaccactgcccatccctccagccaaggaaccaaagtagtgagtgttcaatcgcatcgacaggtttcttagaatatactcaccatccacagtaaactcacgaatcctgtcacaaacaaggagatatggtactacgagattgtcatcaaacccttcacccagcaggtctatccaagcctgcgccctgctcgtttagtaggctatgacggcatctccccaggtcctacgatcatagtgccgagaggaacagaagctgttgtacggtttataaaccagggtgatcgcgaaagctccatccatctccacggctccccctcccgtgccccttttgacggatgggctgatgatatgatcatgaagggggaatacaaaggtacgatagcgtgtgattctacgcatcaggaagcctctatcatactaacaggactttcttctcagactactactacccgaacaaccaagctgccagatttttgtggtaccacgatcatgctatgcatgttgtaagtctttaccgacttttcatggtagtgaaacggaaggattaagctaacatctgtgcagaccgcagaaaatgcctatttcgggcaagccggcgcctacctgatcacagacccggctgaggatgctctcggccttccttcaggttacggaaaatacgacattccgctggtcctcagttccaagtactacaacgccgatggaactcttaagaccagtgtgggagaagacaagagtgtttggggcgacatcatccatgtcaacggtcagccctggccattcttaaatgttgagcctcgaaagtatcgtcttcgattcctcaacgcggctgtttctaggaactttgccctttacttcgtcaagcaagacaacactgccactaggcttcctttccaggtcattgcctctgatgcagggctactcacacacccggttcaaacctcagatatgtatgttgcagccgcagaacgctacgagattgtgttcgatttcgcgccctatgccggccaaacgttggatctgcgcaacttcgcaaaggccaatggtatcggtaccgacgacgactacgcaaacactgacaaggtcatgcgtttccacgtcagcagccaaacagtcgtcgataactccgtggtacccgagcagctatctcagatccagttccccgcggacaaaaccgacatagaccatcacttccgtttccatcgtaccaacggcgagtggcgcatcaacggcatcgggtttgcagacgtcgagaaccgtgttcttgccaaggtaccgcgcggtactgtcgagctttgggaacttgagaacagctccggcggctggtcacaccccatccacgtccacctagtagacttccgagtcgtcgcacgctacggcgacgaaggcactcgcggcgtcatgccctatgaggccgccggtctcaaggacgtcgtgtggctcggccgtcacgagacggtcctcgtcgaagcacattacgccccatgggacggagtctacatgttccactgccacaacctcatccacgaagaccaagacatgatggccgccttcgacgtgactaaactccagaactttgggtacaacgagacgactgatttccacgatcctgaggatcctcgctggtcagcaagacctttcaccgcgggtgatctcacggcgcgatcgggtatcttttcagaagaatccatcagggctagagtaaatgagttggcgctcgagcagccttacagcgaactcgcacaagttacagcctcgctcgagcagtactacaagacgaaccagaaacgccacgacgagtgcgaagacatgcctgctggccctatcccccgttatcgtaggtttcaggtctgattcaagttgttttggtggtgcaacttctccttcttctctccattgaacttaattgtagatgatggatacacactcacttctccctttctatctcgacgctttggccattttatttggtcttattgtgctatatactgtctatttctctttcgtatacgagcaatgtatgtcttggtcggagtcttgtggagctgctgaggtgacacctcgcgacgccatcttagcagttttcgtaactctcgtctatttgtgattactttgttccttaatcagtaacagcttgatgttagattagcaatgagacgaacgatgaagcaatctgagatggatcctttttttttcctaatatttgtatactaaagaatgtgaacaatgccgttttatgaaatgctcataacatgcagcatatttactttgttctatttcatttcattttcatatgtacgcatatcctcggcatcagacaagagacgcgacaacgctctctgcatcccttctcggcccgtaattccgtagaaaatgaccgacgggaaagcagtcctccacgcgctccatgctcatcatgctgcgtactatgtatccccttccaacgcggatggcgcggatgtcgctgcgaacccattgaatgggcatcacgacagccatcatgtcgctaaggacggattcttcttcggatgcaatgcttgtgagggggttttctgcatcccagcaagatgaggtggatcc

SEQ ID NO: 4 - protein, 627 aa, Bipolaris spicifera
MVAKYLFSALQLVSIAKGIYGVALSERPAKFVDNTPDEEKAALASIVEDDPADVVNMLKDWQSPEYPLIFRQPLPIPPAKEPNKLTNPVTNKEIWYYEIVIKPFTQQVYPSLRPARLVGYDGISPGPTIIVPRGTEAVVRFINQGDRESSIHLHGSPSRAPFDGWADDMIMKGEYKDYYYPNNQAARFLWYHDHAMHVTAENAYFGQAGAYLITDPAEDALGLPSGYGKYDIPLVLSSKYYNADGTLKTSVGEDKSVWGDIIHVNGQPWPFLNVEPRKYRLRFLNAAVSRNFALYFVKQDNTATRLPFQVIASDAGLLTHPVQTSDMYVAAAERYEIVFDFAPYAGQTLDLRNFAKANGIGTDDDYANTDKVMRFHVSSQTVVDNSVVPEQLSQIQFPADKTDIDHHFRFHRTNGEWRINGIGFADVENRVLAKVPRGTVELWELENSSGGWSHPIHVHLVDFRVVARYGDEGTRGVMPYEAAGLKDVVWLGRHETVLVEAHYAPWDGVYMFHCHNLIHEDQDMMAAFDVTKLQNFGYNETTDFHDPEDPRWSARPFTAGDLTARSGIFSEESIRARVNELALEQPYSELAQVTASLEQYYKTNQKRHDECEDMPAGPIPRYRRFQV

SEQ ID NO: 5 - DNA, 1791 bp, Artificial Sequence
  cDNA
gtcaatatgctgttcaagtcatggcaactggcagcagcctccgggctcctgtctggagtcctcggcatcccgatggacaccggcagccaccccattgaggctgttgatcccgaagtgaagactgaggtcttcgctgactccctccttgctgcagcaggcgatgacgactgggagtcacctccatacaacttgctttacaggaatgccctgccaattccacctgtcaagcagcccaagatgatcattaccaaccctgtcaccggcaaggacatttggtactatgagatcgagatcaagccatttcagcaaaggatttaccccaccttgcgccctgccactctcgtcggctacgatggcatgagccctggtcctactttcaatgttcccagaggaacagagactgtagttaggttcatcaacaatgccaccgtggagaactcggtccatctgcacggctccccatcgcgtgcccctttcgatggttgggctgaagatgtgaccttccctggcgagtacaaggattactactttcccaactaccaatccgcccgccttctgtggtaccatgaccacgctttcatgaagactgctgagaatgcctactttggtcaggctggcgcctacattatcaacgacgaggctgaggatgctctcggtcttcctagtggctatggcgagttcgatatccctctgatcctgacggccaagtactataacgccgatggtaccctgcgttcgaccgagggtgaggaccaggacctgtggggagatgtcatccatgtcaacggacagccatggcctttccttaacgtccagccccgcaagtaccgtttccgattcctcaacgctgccgtgtctcgtgcttggctcctctacctcgtcaggaccagctctcccaacgtcagaattcctttccaagtcattgcctctgatgctggtctccttcaagcccccgttcagacctctaacctctaccttgctgttgccgagcgttacgagatcattattgacttcaccaactttgctggccagactcttgacctgcgcaacgttgctgagaccaacgatgtcggcgacgaggatgagtacgctcgcactctcgaggtgatgcgcttcgtcgtcagctctggcactgttgaggacaacagccaggtcccctccactctccgtgacgttcctttccctcctcacaaggaaggccccgccgacaagcacttcaagtttgaacgcagcaacggacactacctgatcaacgatgttggctttgccgatgtcaatgagcgtgtcctggccaagcccgagctcggcaccgttgaggtctgggagctcgagaactcctctggaggctggagccaccccgtccacattcaccttgttgacttcaagatcctcaagcgaactggtggtcgtggccaggtcatgccctacgagtctgctggtcttaaggatgtcgtctggttgggcaggggtgagaccctgaccatcgaggcccactaccaaccctggactggagcttacatgtggcactgtcacaacctcattcacgaggataacgacatgatggctgtattcaacgtcaccgccatggaggagaagggatatcttcaggaggacttcgaggaccccatgaaccccaagtggcgcgccgttccttacaaccgcaacgacttccatgctcgcgctggaaacttctccgccgagtccatcactgcccgagtgcaggagctggccgagcaggagccgtacaaccgcctcgatgagatcctggaggatcttggaatcgaggagtaa

SEQ ID NO: 6 - DNA, 2063 bp, Curvularia pallescens
atggttgccaaatacctcttctcggcacttcaactcgcttcaattgcgaaaggcatatacggcgttgctttgagcgagcgtcctgccaaatatattgacgaaacccccgacgaagaaaaggctgccctggcagccatcgttgaagatgaccctgccgatgttttcagaatcctgaaggactggcaaagcccggagtatcccatcctttttcgcgaggcactgcccatccctccagccaaggaaccgaagtagtgagtcttgaattgcatggacaggtttcctagaatatgctcacccatccgcagtaaaatgacgaatcctgtcacaaacaaggagatctggtactacgagattgtcatcaaaccctttaaccaacaggtctatccaagtctacgtcctgctcgcttggtaggctatgatggcatttcaccaggccctacgatcatcgtgccgagaggaacagaagccgttgtacgattcgtaaaccagggtgatcgcgagagttcgattcatcttcatggttctccctcccgtgccccctttgacggatgggctgaagatttgattatgaagggccaattcaaaggtacaacagaacaatcttatgcatcagggtgcctcttttatactaacacgactcgttcttagactactactacccgaacaaccaggctgccagattcctgtggtaccacgatcatgctatgcatgttgtaagtcttgcagactaatcatgggagcgaaacggaaagatcgggctgacacttatgcagactgcggaaaatgcctattttggacaggctggcgcctacctgatcacagacccagctgaggacgccctcggccttccttcgggttacggaaaatacgacatcccactggtgctcagttccaagttctacaacagtgatggaactctccagaccagtgtgggagaagacaacagtctctggggcgacgtcatccatgtcaacggtcagccctggccattcttcaacgttgagcctcgaaagtatcgccttcgattcctcaatgcggctgtttctcggaactttgccctctatttcgtcaagcaacaagccactgctactagacttcctttccaggtcattgcctctgatgcagggctactcacgcacccggtccaaacctcagatatttacgtggcagcagcagagcgctacgagattgtattcgactttgcgccttatgcaggccagacgatagatttgcgtaactttgcaaaggccaatggggtcggcaccgatgacgattatgcaaacactgacaaggtcatgcgcttccatgtcagcagccaagcagtcgtcgataactcggtggtacccgcacagctatctcagatccagttccccgccgacaaaaccggcatcgaccaccacttccgcttccatcgcaccaacagcgagtggcgcatcaacggcatcgggtttgcagacgtccagaaccgtatcctggccaaggtaccgcgcggcactgtcgagctatgggaactcgagaacagctccggcggctggtcgcaccccatccacgtccacctggtcgacttccgagtcgtcgcacgctacggtgacgaaagcactcgcggcgtcatgccctacgagtccgccggtctcaaggacgtcgtgtggctcggccgccacgagacggtgctcgtcgaagcacactacgccccctgggacggagtctacatgttccactgccacaacctgatccacgaagaccaagacatgatggccgcgtttgacgtgactaagctccagaactttggctacaacgagacgacggatttccacgacccggaagattctcgctggtctgcaagacccttcaccgcggctgacttgacggcgcgatcgggtatcttctcagaagcatccatcagggctagagtgaacgagttggcgctggaacagccgtacagcgaactggcacaggtcacggcctcgctcgagcagtactacaagacgaacaagaaacgccaggccgagtgcgaagacatgcctgctggccccattccccgttatcgcaggtttcaggtctga

SEQ ID NO: 7 - protein, 627 aa, Curvularia pallescens
MVAKYLFSALQLASIAKGIYGVALSERPAKYIDETPDEEKAALAAIVEDDPADVFRILKDWQSPEYPILFREALPIPPAKEPNKMTNPVTNKEIWYYEIVIKPFNQQVYPSLRPARLVGYDGISPGPTIIVPRGTEAVVRFVNQGDRESSIHLHGSPSRAPFDGWAEDLIMKGQFKDYYYPNNQAARFLWYHDHAMHVTAENAYFGQAGAYLITDPAEDALGLPSGYGKYDIPLVLSSKFYNSDGTLQTSVGEDNSLWGDVIHVNGQPWPFFNVEPRKYRLRFLNAAVSRNFALYFVKQQATATRLPFQVIASDAGLLTHPVQTSDIYVAAAERYEIVFDFAPYAGQTIDLRNFAKANGVGTDDDYANTDKVMRFHVSSQAVVDNSVVPAQLSQIQFPADKTGIDHHFRFHRTNSEWRINGIGFADVQNRILAKVPRGTVELWELENSSGGWSHPIHVHLVDFRVVARYGDESTRGVMPYESAGLKDVVWLGRHETVLVEAHYAPWDGVYMFHCHNLIHEDQDMMAAFDVTKLQNFGYNETTDFHDPEDSRWSARPFTAADLTARSGIFSEASIRARVNELALEQPYSELAQVTASLEQYYKTNKKRQAECEDMPAGPIPRYRRFQV

SEQ ID NO: 8 - DNA, 858 bp, Amerosporium atrum
  misc_feature (1)...(858): n = A,T,C or G
caccgccgagaacgcttactttggtcaagctggcttttacattctgcacgaccccgctgaagatgcattgggtctgccttctggcaagtatgatgtacctcttgcactgtcctccaagcagtacaacagcgacggtaccctcttcgaccccaaggacgagaccgattcactgttcggcgatgtcatccacgtcaacggacagccatggccctactttaaggtcgagcctcgcaagtaccgtctccgcttcctcaatgctgctatcagccgtgccttcaagctcactttcgaggctgatggcaaagtgatcaactttcctgtcatcggtgccgatactggtctcttgaccaagcctgttcagacaagcaaccttgagatctctatggccgagcgctgggaggttgtttttgacttcagccaattttccgggaagaacgtcaccctcaagaacggtcgcgatgtgcagcacgatgaggactacaactccaccgacaaagtcatgcagttcgttgttggcaaggatgttacgagccaggctggtaatggcaaccttcccggctctctgcgcactgttcccttccctcctaagaaggggcggagtcgacaggagcttcaagttcggcagggaccggtggccagtggactgttaatggcttgaccttcgctgatgtcaacaaccgcatcctggctaagcccccaacgtggtgccatcgaggttttgggagctttgagaacttccagcggnggntggtcttacccttgtccacatccacctgggtcgactttccagatncttgtcttgcactggangcaaggcnccccgttntaactncnanaaaggaagcactttcaagggcg

SEQ ID NO: 9 - protein, 114 aa, Amerosporium atrum
  VARIANT (1)...(114): Xaa = space of unknown number of aa
TAENAYFGQAGFYILHDPAEDALGLPSGKYDVPLALSLKAYNSDGTLFDPKDETDSLFGDVIHVNGQPWPYLKVEPRKYRLRFLNAAISRAFKXVWELENTSSGGWSYPVHIHL

SEQ ID NO: 10 - protein, 19 aa, Stachybotrys chartarum
  VARIANT (1)...(19): Xaa = Any Amino Acid
DYYFPNYQSARLLXYHDHA

SEQ ID NO: 11 - protein, 13 aa, Stachybotrys chartarum
RGQVMPYESAGLK

SEQ ID NO: 12 - DNA, 20 bp, Artificial Sequence
  degenerated primer
tattactttccnaantanca

SEQ ID NO: 13 - DNA, 20 bp, Artificial Sequence
  degenerated primer
tcgtatggcatnacctgncc

SEQ ID NO: 14 - DNA, 18 bp, Artificial Sequence
  oligonucleotide primer
tggtaccangancangct

SEQ ID NO: 15 - DNA, 18 bp, Artificial Sequence
  oligonucleotide primer
ngactcgtanggcatgac

SEQ ID NO: 16 - DNA, 21 bp, Artificial Sequence
  oligonucleotide primer
tcgtggatgannttgtgncan

SEQ ID NO: 17 - DNA, 21 bp, Artificial Sequence
  oligonucleotide primer
cnagacnacntcnttnagacc

(19) United States
(12) Patent Application Publication
(10) Pub. No.: US 2006/0024784 A1
(43) Pub. Date: Feb. 2, 2006

(54) Phenol oxidizing enzymes

(76) Inventors: Huaming Wang, Fremont, CA (US); Elizabeth A. Bodie, San Carlos, CA (US)

Correspondence Address: Genencor International, Inc., 925 Page Mill Road, Palo Alto, CA, 94034-1013, US

(21) Appl. No.: 11/130,559

(22) Filed: May 17, 2005

Related U.S. Application Data
(63) Continuation of application No. 10/080,210, filed on Feb. 19, 2002, now abandoned.
Application No. 10/080,210 is a continuation of application No. 09/468,578, filed on Dec. 21, 1999, now Pat. No. 6,399,329.
Application No. 09/468,578 is a continuation-in-part of application No. 09/338,723, filed on Jun. 23, 1999, now abandoned.
Application No. 09/338,723 is a continuation of application No. 09/220,871, filed on Dec. 23, 1998, now abandoned.

(51) Int. Cl. C12P 21/06; C07H 21/04; C12N 9/02; C12N 15/74; C12N 1/16

(52) U.S. Cl. 435/69.1; 435/189; 435/254.1; 435/484; 536/23.2

(57) ABSTRACT

Disclosed herein are novel phenol oxidizing enzymes encoded by nucleic acid capable of hybridizing to the nucleic acid having the sequence as shown in SEQ ID NO:1 and in particular those obtainable from fungus. The present invention provides nucleic acid sequences and amino acid sequences from Bipolaris spicifera, Curvularia pallescens and Amerosporium atrum. The present invention provides expression vectors and host cells comprising nucleic acid encoding phenol oxidizing enzymes, methods for producing the phenol oxidizing enzyme as well as methods for constructing expression hosts.

### FIELD OF THE INVENTION

The present invention relates to novel phenol oxidizing enzymes, in particular, novel phenol oxidizing enzymes obtainable from fungus. The present invention provides methods and host cells for expressing the phenol oxidizing enzymes as well as methods for producing expression systems comprising the phenol oxidizing enzymes.

### BACKGROUND OF THE INVENTION

Phenol oxidizing enzymes function by catalyzing redox reactions, i.e., the transfer of electrons from an electron donor (usually a phenolic compound) to molecular oxygen (which acts as an electron acceptor) which is reduced to H2O. While being capable of using a wide variety of different phenolic compounds as electron donors, phenol oxidizing enzymes are very specific for molecular oxygen as the electron acceptor.

Phenol oxidizing enzymes can be utilized for a wide variety of applications, including the detergent industry, the paper and pulp industry, the textile industry and the food industry. In the detergent industry, phenol oxidizing enzymes have been used for preventing the transfer of dyes in solution from one textile to another during detergent washing, an application commonly referred to as dye transfer inhibition. Most phenol oxidizing enzymes exhibit pH optima in the acidic pH range while being inactive in neutral or alkaline pHs.

Phenol oxidizing enzymes are known to be produced by a wide variety of fungi, including species of the genii Aspergillus, Neurospora, Podospora, Botytis, Pleurotus, Fomes, Phlebia, Trametes, Polyporus, Rhizoctonia and Lentinus. However, there remains a need to identify and isolate phenol oxidizing enzymes, and organisms capable of naturally-producing phenol oxidizing enzymes for use in textile, cleaning and detergent washing methods and compositions.

### SUMMARY OF THE INVENTION

The present invention relates to novel phenol oxidizing enzymes encoded by nucleic acid capable of hybridizing to the nucleic acid encoding Stachybotrys chartarum phenol oxidizing enzyme (shown in FIG. 1, and having the polynucleotide sequence shown in SEQ ID NO:1), or a fragment thereof, under conditions of high to intermediate stringency, as long as the phenol oxidizing enzyme is capable of modifying the color associated with dyes or colored compounds. In illustrative embodiments disclosed herein, the phenol oxidizing enzymes are obtainable from fungus. The phenol oxidizing enzymes of the present invention can be used, for example, for pulp and paper bleaching, for bleaching the color of stains on fabric and for anti-dye transfer in detergent and textile applications. The phenol oxidizing enzymes of the present invention may be capable of modifying the color in the absence of an enhancer or in the presence of an enhancer.

Accordingly, the present invention provides phenol oxidizing enzymes encoded by nucleic acid capable of hybridizing to the nucleic acid having the sequence as shown in SEQ ID NO:1 or a fragment thereof, under conditions of intermediate to high stringency. Such enzymes will comprise at least 60% identity, at least 65% identity, at least 70% identity, at least 75% identity, at least 80% identity, at least 85% identity, at least 90% identity and at least 95% identity to the Stachybotrys chartarum phenol oxidizing enzyme having the amino acid sequence disclosed in SEQ ID NO:2, and specifically excludes the amino acid sequence shown in SEQ ID NO:2, as long as the enzyme is capable of modifying the color associated with dyes or colored compounds. In one embodiment, the phenol oxidizing enzyme is obtainable from bacteria, yeast or non-Stachybotrys species of fungus. In a preferred embodiment, the phenol oxidizing enzyme is obtainable from fungus including Myrothecium species, Curvularia species, Chaetomium species, Bipolaris species, Humicola species, Pleurotus species, Trichoderma species, Mycellophthora species and Amerosporium species. In a preferred embodiment, the fungus include Myrothecium verrucaria, Curvularia pallescens, Chaetomium sp, Bipolaris spicifera, Humicola insolens, Pleurotus abalonus, Trichoderma reesei, Mycellophthora thermophila and Amerosporium atrum.

In an illustrative embodiment disclosed herein, the phenol oxidizing enzyme is obtainable from Bipolaris spicifera and has the genomic nucleic acid sequence as shown in FIG. 2 (SEQ ID NO:3) and the deduced amino acid sequence as shown in FIG. 3 (SEQ ID NO:4). In another illustrative embodiment disclosed herein, the phenol oxidizing enzyme is obtainable from Curvularia pallescens and has the genomic nucleic acid sequence as shown in FIG. 9 (SEQ ID NO:6) and the deduced amino acid sequence as shown in FIG. 10 (SEQ ID NO:7). In another illustrative embodiment disclosed herein, the phenol oxidizing enzyme is obtainable from Amerosporium atrum and comprises the nucleic acid sequence as shown in FIG. 13 (SEQ ID NO: 8) and the deduced amino acid sequence as shown in FIG. 13 (SEQ ID NO:9).

Accordingly, the present invention encompasses phenol oxidizing enzymes encoded by polynucleotide sequences that hybridize under conditions of intermediate to high stringency to the nucleic acid having the sequence as shown in SEQ ID NO:3, SEQ ID NO:6 or SEQ ID NO:8, or a fragment thereof, and which are capable of modifying the color associated with a dye or colored compound. The present invention also encompasses polynucleotides that encode the amino acid sequence as shown in SEQ ID NO:4 as well as polynucleotides that encode the amino acid sequence as shown in SEQ ID NO:7 and polynucleotides that encode the amino acid sequence as shown in SEQ ID NO:9. The present invention provides expression vectors and host cells comprising polynucleotides encoding the phenol oxidizing enzymes of the present invention as well as methods for producing the enzymes.

The present invention provides a method for producing a phenol oxidizing enzyme comprising the steps of obtaining a host cell comprising a polynucleotide capable of hybridizing to SEQ ID NO:1, or a fragment thereof, under conditions of intermediate to high stringency wherein said polynucleotide encodes a phenol oxidizing enzyme capable of modifying the color associated with dyes or colored compounds; growing said host cell under conditions suitable for the production of said phenol oxidizing enzyme; and optionally recovering said phenol oxidizing enzyme produced. In one embodiment, the polynucleotide comprises the sequence as shown in SEQ ID NO:3; in another embodiment, the polynucleotide comprises the sequence as shown in SEQ ID NO:6; and in another embodiment, the polynucleotide comprises the sequence as shown in SEQ ID NO: 8. In another embodiment, the phenol oxidizing enzyme comprises the amino acid sequence as shown in SEQ ID NO:4; in a further embodiment, the phenol oxidizing enzyme comprises the amino acid sequence as shown in SEQ ID NO:7; and in yet another embodiment, the phenol oxidizing enzyme comprises the amino acid sequence as shown in SEQ ID NO:9.

The present invention also provides a method for producing a host cell comprising a polynucleotide encoding a phenol oxidizing enzyme comprising the steps of obtaining a polynucleotide capable of hybridizing to SEQ ID NO:1, or fragment thereof, under conditions of intermediate to high stringency wherein said polynucleotide encodes a phenol oxidizing enzyme capable of modifying the color associated with dyes or colored compounds; introducing said polynucleotide into said host cell; and growing said host cell under conditions suitable for the production of said phenol oxidizing enzyme. In one embodiment, the polynucleotide comprises the sequence as shown in SEQ ID NO:3. In another embodiment, the polynucleotide comprises the sequence as shown in SEQ ID NO:6. In a further embodiment, the polynucleotide comprises the sequence as shown in SEQ ID NO:8. In the present invention, the host cell comprising a polynucleotide encoding a phenol oxidizing enzyme includes filamentous fungus, yeast and bacteria. In one embodiment, the host cell is a filamentous fungus including Aspergillus species, Trichoderma species and Mucor species. In a further embodiment, the filamentous fungus host cell includes Aspergillus niger var. awamori or Trichoderma reesei.

In yet another embodiment of the present invention, the host cell is a yeast which includes Saccharomyces, Pichia, Hansenula, Schizosaccharomyces, Kluyveromyces and Yarrowia species. In an additional embodiment, the Saccharomyces species is Saccharomyces cerevisiae. In yet an additional embodiment, the host cell is a gram positive bacteria, such as a Bacillus species, or a gram negative bacteria, such as an Escherichia species.

Also provided herein are detergent compositions comprising a phenol oxidizing enzyme encoded by nucleic acid capable of hybridizing to the nucleic acid encoding Stachybotrys chartarum phenol oxidizing enzyme (shown in FIG. 1 and having SEQ ID NO:1) under conditions of intermediate to high stringency. Such enzymes will have at least 60% identity, at least 65% identity, at least 70% identity, at least 75% identity, at least 80% identity, at least 85% identity, at least 90% identity and at least 95% identity to the phenol oxidizing enzyme having the amino acid sequence disclosed in SEQ ID NO:2, and will specifically exclude the amino acid having the sequence as shown in SEQ ID NO:2, as long as the enzyme is capable of modifying the color associated with dyes or colored compounds. In one embodiment of the detergent composition, the amino acid comprises the sequence as shown in SEQ ID NO:4. In another embodiment of the detergent composition, the amino acid comprises the sequence as shown in SEQ ID NO:7. In a further embodiment of the detergent composition, the amino acid comprises the sequence as shown in SEQ ID NO:9.

The present invention also encompasses methods for modifying the color associated with dyes or colored compounds which occur in stains in a sample, comprising the steps of contacting the sample with a composition comprising a phenol oxidizing enzyme encoded by nucleic acid capable of hybridizing to the nucleic acid encoding Stachybotrys chartarum phenol oxidizing enzyme (shown in FIG. 1 and having SEQ ID NO:1) under conditions of intermediate to high stringency. Such phenol oxidizing enzymes will have at least 60% identity, at least 65% identity, at least 70% identity, at least 75% identity, at least 80% identity, at least 85% identity, at least 90% identity and at least 95% identity to the phenol oxidizing enzyme having the amino acid sequence disclosed in SEQ ID NO:2, and specifically excludes the amino acid having the sequence as shown in SEQ ID NO:2, as long as the enzyme is capable of modifying the color associated with dyes or colored compounds. In one embodiment of the method, the amino acid comprises the amino acid sequence as shown in SEQ ID NO:4. In another embodiment, the amino acid comprises the amino acid sequence as shown in SEQ ID NO:7. In a further embodiment, the amino acid comprises the amino acid having the sequence as shown in SEQ ID NO:9.

### BRIEF DESCRIPTION OF THE DRAWINGS

FIG. 1 provides the genomic nucleic acid sequence (SEQ ID NO:1) encoding a phenol oxidizing enzyme obtainable from Stachybotrys chartarum.

FIG. 2 provides the genomic sequence (SEQ ID NO:3) encoding a phenol oxidizing enzyme obtainable from Bipolarius spicifera.

FIG. 3 provides the deduced amino acid sequence (SEQ ID NO:4) for a phenol oxidizing enzyme obtainable from Bipolarius spicifera.

FIG. 4 is an amino acid alignment of phenol oxidizing enzyme obtainable from Stachybotrys chartarum SEQ ID NO:2 (top line) and Bipolarius spicifera (SEQ ID NO:4).

FIG. 5 is a cDNA (SEQ ID NO:5) and amino acid sequence (SEQ ID NO:2) obtainable from Stachybotrys chartarum.

FIG. 6 is a representation of the Southern hybridization technique described in Example IV. The genomic DNA was isolated from following strains: Stachybotrys chartarum (lanes 1 and 2), Myrothecium verruvaria (lanes 3 and 4), Curvalaria pallescens (lanes 5 and 6), Myrothecium cinctum (lanes 7 and 8), Pleurotus eryngii (lanes 9 and 10), Humicola insulas (lanes 11 and 12). The genomic DNA was digested with restriction enzymes EcoRI (lanes 1, 3, 5, 7, 9, 11) or HindIII (lanes 2, 4, 6, 8, 10 and 12). The DNA probe used for Southern analysis was isolated from a Stachybotrys chartarum genomic fragment generated through PCR that covers the internal part of the genes of more than 1 kb in size. The same DNA probe was used in the Southern hybridization techniques illustrated in FIGS. 7, 8 and 9.

FIG. 7 is a representation of the Southern hybridization technique described in Example IV. The genomic DNA was isolated from following strains: Stachybotrys chartarum (lanes 1 and 2), Aspergillus niger (lanes 3 and 4), Corpinus cineras (lanes 5 and 6), Mycellophthora thermophila (lanes 7 and 8), Pleurotus abalonus (lanes 9 and 10), Trichoderma reesei (lanes 11 and 12). The genomic DNA was digested with restriction enzymes EcoRI (lanes 1, 3, 5, 7, 9, 11) or HindIII (lanes 2, 4, 6, 8, 10 and 12).

FIG. 8 is a representation of the Southern hybridization technique described in Example IV. The genomic DNA was isolated from following strains: Stachybotrys chartarum (lane 1); Trametes vesicolor (lanes 2 and 3); Amerosporium atrum (lanes 6 and 7); Bipolaris spicifera (lanes 8 and 9); Chaetomium sp (lanes 10 and 11). The genomic DNA was digested with restriction enzymes EcoRI (lanes 1, 2, 8 and 10) or HindIII (lanes 3, 9 and 11).

FIG. 9 provides the genomic nucleic acid sequence of a phenol oxidizing enzyme obtainable from Curvularia pallescens from the translation start site to the translation stop site.

FIG. 10 provides the deduced amino acid sequence of the phenol oxidizing enzyme obtainable from Curvularia pallescens.

FIG. 11 provides an amino acid alignment between the amino acid sequence obtainable from Bipolaris spicifera shown in SEQ ID NO:4 (bottom line) and Curvularia pallescens shown in SEQ ID NO:7 (top line).

FIG. 12 shows the Bipolaris spicifera pH profile as measured at 470 nm using Guaicol as a substrate.

FIG. 13 shows the Amerosporium atrum nucleic acid (SEQ ID NO:8) and deduced amino acid sequence (SEQ ID NO:9).

FIG. 14 provides an amino acid alignment between the amino acid sequence obtainable from Amerosporium atrum (SEQ ID NO:9) (bottom line) and the amino acid sequence obtainable from Stachybotrys chartarum (SEQ ID NO:2) (top line).

### DETAILED DESCRIPTION

Definitions

As used herein, the term “phenol oxidizing enzyme” refers to those enzymes which catalyze redox reactions and are specific for molecular oxygen and/or hydrogen peroxide as the electron acceptor. The phenol oxidizing enzymes described herein are encoded by nucleic acid capable of hybridizing to SEQ ID NO:1 (which encodes a phenol oxidizing enzyme obtainable from Stachybotrys chartarum ATCC number 38898), or a fragment thereof, under conditions of intermediate to high stringency and are capable of modifying the color associated with a dye or colored compound. Such phenol oxidizing enzymes will have at least 60% identity, at least 65% identity, at least 70% identity, at least 75% identity, at least 80% identity, at least 85% identity, at least 90% identity and at least 95% identity to the phenol oxidizing enzyme having the amino acid sequence disclosed in SEQ ID NO:2 as determined by MegAlign Program from DNAstar (DNASTAR, Inc. Madison, Wis. 53715) by Jotun Hein Method (1990, Method in Enzymology, 183: 626-645).

As used herein, Stachybotrys refers to any Stachybotrys species which produces a phenol oxidizing enzyme capable of modifying the color associated with dyes or colored compounds. The present invention encompasses derivatives of natural isolates of Stachybotrys, including progeny and mutants, as long as the derivative is able to produce a phenol oxidizing enzyme capable of modifying the color associated with dye or color compounds.

As used herein in referring to phenol oxidizing enzymes, the term “obtainable from” means phenol oxidizing enzymes equivalent to those that originate from or are naturally-produced by the particular microbial strain mentioned. To exemplify, phenol oxidizing enzymes obtainable from Bipolaris refer to those phenol oxidizing enzymes which are naturally-produced by Bipolaris. The present invention encompasses phenol oxidizing enzymes produced recombinantly in host organisms through genetic engineering techniques. For example, a phenol oxidizing enzyme obtainable from Bipolaris can be produced in an Aspergillus species through genetic engineering techniques.

As used herein, the term ‘colored compound’ refers to a substance that adds color to textiles or to substances which result in the visual appearance of stains. As defined in Dictionary of Fiber and Textile Technology (Hoechst Celanese Corporation (1990) PO Box 32414, Charlotte N.C. 28232), a dye is a colored compound that is incorporated into the fiber by chemical reaction, absorption, or dispersion. Examples of dyes include direct Blue dyes, acid Blue dyes, direct red dyes, reactive Blue and reactive Black dyes. A catalogue of commonly used textile dyes is found in Colour Index, 3rd ed. Vol. 1-8. Examples of substances which result in the visual appearance of stains are polyphenols, carotenoids, anthocyanins, tannins, Maillard reaction products, etc.

As used herein the phrase “modify the color associated with a dye or colored compound” or “modification of the colored compound” means that the dye or compound is changed through oxidation such that either the color appears modified, i.e., the color visually appears to be decreased, lessened, decolored, bleached or removed, or the color is not affected but the compound is modified such that dye redeposition is inhibited. The present invention encompasses the modification of the color by any means including, for example, the complete removal of the colored compound from stain on a sample, such as a fabric, by any means as well as a reduction of the color intensity or a change in the color of the compound. For example, in pulp and paper applications, delignification in the pulp results in higher brightness in paper made from the pulp.

As used herein, the term “mutants and variants”, when referring to phenol oxidizing enzymes, refers to phenol oxidizing enzymes obtained by alteration of the naturally occurring amino acid sequence and/or structure thereof, such as by alteration of the nucleic acid sequence of the structural gene and/or by direct substitution and/or alteration of the amino acid sequence and/or structure of the phenol oxidizing enzyme. The term phenol oxidizing enzyme “derivative” as used herein refers to a portion or fragment of the full-length naturally occurring or variant phenol oxidizing enzyme amino acid sequence that retains at least one activity of the naturally occurring phenol oxidizing enzyme. As used herein, the term “mutants and variants”, when referring to microbial strains, refers to cells that are changed from a natural isolate in some form, for example, having altered DNA nucleotide sequence of, for example, the structural gene coding for the phenol oxidizing enzyme; alterations to a natural isolate in order to enhance phenol oxidizing enzyme production; or other changes that effect phenol oxidizing enzyme expression.

The term “enhancer” or “mediator” refers to any compound that is able to modify the color associated with a dye or colored compound in association with a phenol oxidizing enzyme or a compound which increases the oxidative activity of the phenol oxidizing enzyme. The enhancing agent is typically an organic compound.

Phenol Oxidizing Enzymes

The phenol oxidizing enzymes of the present invention function by catalyzing redox reactions, i.e., the transfer of electrons from an electron donor (usually a phenolic compound) to molecular oxygen and/or hydrogen peroxide (which acts as an electron acceptor) which is reduced to water. Examples of such enzymes are laccases (EC 1.10.3.2), bilirubin oxidases (EC 1.3.3.5), phenol oxidases (EC 1.14.18.1), catechol oxidases (EC 1.10.3.1).

The present invention encompasses phenol oxidizing enzymes obtainable from bacteria, yeast or non-Stachybotrys fungal species said enzymes being encoded by nucleic acid capable of hybridizing to the nucleic acid as shown in SEQ ID NO:1 under conditions of intermediate to high stringency, as long as the enzyme is capable of modifying the color associated with a dye or colored compound.

Phenol oxidizing enzymes encoded by nucleic acid capable of hybridizing to SEQ ID NO:1, or a fragment thereof, are obtainable from bacteria, yeast and non-Stachybotrys fungal species including, but not limited to Myrothecium verrucaria, Curvalaria pallescens, Chaetomium sp, Bipolaris spicifera, Humicola insolens, Pleurotus abalonus, Trichoderma reesei, Mycellophthora thermophila and Amerosporium atrum. Illustrative examples of isolated and characterized phenol oxidizing enzymes encoded by nucleic acid capable of hybridizing to SEQ ID NO:1 are provided herein and include phenol oxidizing enzymes obtainable from strains of Bipolaris spicifera, Curvularia pallescens, and Amerosporium atrum and include the phenol oxidizing enzymes comprising the amino acid sequences as shown in SEQ ID NO: 4, SEQ ID NO:7, and SEQ ID NO: 9, respectively. The amino acid sequence shown in SEQ ID NO:9 represents a partial amino acid sequence.

Strains of Bipolaris spicifera are available from the Centraalbureau Voor Schimmelcultures Baarn (CBS)-Delft (The Netherlands) Institute of the Royal Netherlands Academy of Arts and Sciences and have CBS accession number CBS 197.31; CBS 198.31; CBS 199.31; CBS 211.34; CBS 274.52; CBS 246.62; CBS 314.64; CBS 315.64; CBS 418.67; CBS 364.70 and CBS 586.80.

Strains of Curvularia pallescens are available from the American Type Culture Collection (ATCC) and include ATCC accession numbers ATCC 12018; ATCC 22920; ATCC 32910; ATCC 34307; ATCC 38779; ATCC 44765; ATCC 60938; ATCC 60939; and ATCC 60941.

Strains of Amerosporium atrum are available from the CBS and include CBS accession numbers, CBS 142.59; CBS 166.65; CBS 151.69; CBS 548.86.

As will be understood by the skilled artisan, there may be slight amino acid variations of the phenol ozidizing enzyme found among the variety of deposited strains of a particular organism. For example, among the variety of Bipolaris spicifera strains deposited with the CBS, there may be amino acid sequences having 95% or greater identity to the amino acid sequence shown in SEQ ID NO:4 and similarly, among the variety of Curvularia pallescens strains deposited with the ATCC, there may be amino acid sequences having 95% or greater identity to the amino acid sequence shown in SEQ ID NO:7. Additionally, among the variety of Amerosporium atrum strains deposited with the CBS, there may be amino acid sequences having 95% or greater identity to the amino acid sequence shown in SEQ ID NO:9. Therefore, the present invention encompasses phenol oxidizing enzymes obtainable from strains of Bipolaris spicifera that have at least 95% identity to the amino acid sequence shown in SEQ ID NO:4. The present invention also encompasses phenol oxidizing enzymes obtainable from strains of Curvularia pallescens that have at least 95% identity to the amino acid sequence shown in SEQ ID NO:7. The present invention also encompasses phenol oxidizing enzymes obtainable from strains of Amerosporium atrum that have at least 95% identity to the amino acid sequence shown in SEQ ID NO:9.

Nucleic Acid Encoding Phenol Oxidizing Enzymes

The present invention encompasses polynucleotides which encode phenol oxidizing enzymes obtainable from bacteria, yeast or non-Stachybotrys fungal species which polynucleotides comprise at least 60% identity, at least 65% identity, at least 70% identity, at least 75% identity, at least 80% identity, at least 85% identity, at least 90% identity and at least 95% identity to the polynucleotide sequence disclosed in SEQ ID NO:1 (as determined by MegAlign Program from DNAstar (DNASTAR, Inc. Maidson, Wis. 53715) by Jotun Hein Method (1990, Method in Enzymology, 183: 626-645) with a gap penalty=11, a gap length penalty=3 and Pairwise Alignment Parameters Ktuple=2) as long as the enzyme encoded by the polynucleotide is capable of modifying the color associated with dyes or colored compounds. In a preferred embodiment, the phenol oxidizing enzyme is encoded by a polynuleotide comprising the sequence as shown in SEQ ID NO:3. In another preferred embodiment, the phenol oxidizing enzyme is encoded by a polynucleotide comprising the sequence as shown in SEQ ID NO:6. In yet another preferred embodiment, the phenol oxidizing enzyme is encoded by the polynucleotide comprising the sequence as shown in SEQ ID NO:8. As will be understood by the skilled artisan, due to the degeneracy of the genetic code, a variety of polynucleotides can encode the phenol oxidizing enzyme disclosed in SEQ ID NO:4, SEQ ID NO:7 and SEQ ID NO:9. The present invention encompasses all such polynucleotides.

The nucleic acid encoding a phenol oxidizing enzyme may be obtained by standard procedures known in the art from, for example, cloned DNA (e.g., a DNA “library”), by chemical synthesis, by cDNA cloning, by PCR, or by the cloning of genomic DNA, or fragments thereof, purified from a desired cell, such as a Biopolaris species, Curvularia species or Amerosporium species (See, for example, Sambrook et al., 1989, Molecular Cloning, A Laboratory Manual, 2d Ed., Cold Spring Harbor Laboratory Press, Cold Spring Harbor, N.Y. Glover, D. M. (ed.), 1985, DNA Cloning: A Practical Approach, MRL Press, Ltd., Oxford, U.K. Vol. I, II.). Nucleic acid sequences derived from genomic DNA may contain regulatory regions in addition to coding regions. Whatever the source, the isolated nucleic acid encoding a phenol oxidizing enzyme of the present invention should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis, PCR and column chromatography.

Once nucleic acid fragments are generated, identification of the specific DNA fragment encoding a phenol oxidizing enzyme may be accomplished in a number of ways. For example, a phenol oxidizing enzyme encoding gene of the present invention or its specific RNA, or a fragment thereof, such as a probe or primer, may be isolated and labeled and then used in hybridization assays to detect a generated gene. (Benton, W. and Davis, R., 1977, Science 196:180; Grunstein, M. And Hogness, D., 1975, Proc. Natl. Acad. Sci. USA 72:3961). Those DNA fragments sharing substantial sequence similarity to the probe will hybridize under stringent conditions.

The present invention encompasses phenol oxidizing enzymes encoded by nucleic acid identified through nucleic acid hybridization techniques using SEQ ID NO:1 as a probe or primer and screening nucleic acid of either genomic or cDNA origin. Nucleic acid encoding phenol oxidizing enzymes obtainable from bacteria, yeast or non-Stachybotrys fungal species and having at least 60% identity to SEQ ID NO:1 can be detected by DNA-DNA or DNA-RNA hybridization or amplification using probes, portions or fragments of SEQ ID NO:1. Accordingly, the present invention provides a method for the detection of nucleic acid encoding a phenol oxidizing enzyme encompassed by the present invention which comprises hybridizing part or all of a nucleic acid sequence of SEQ ID NO:1 with Stachybotrys nucleic acid of either genomic or cDNA origin.

Also included within the scope of the present invention are polynucleotide sequences that are capable of hybridizing to the nucleotide sequence disclosed in SEQ ID NO:1 under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.) incorporated herein by reference, and confer a defined “stringency” as explained below.

“Maximum stringency” typically occurs at about Tm-5° C. (5° C. below the Tm of the probe); “high stringency” at about 5° C. to 10° C. below Tm; “intermediate stringency” at about 10° C. to 20° C. below Tm; and “low stringency” at about 20° C. to 25° C. below Tm. For example in the present invention the following are the conditions for high stringency: hybridization was done at 37° C. in buffer containing 50% formamide, 5×SSPE, 0.5% SDS and 50 ug/ml of sheared Herring DNA. The washing was performed at 65° C. for 30 minutes in the presence of 1×SSC and 0.1% SDS once, at 65° C. for 30 minutes in presence of 0.5×SSC and 0.1% SDS once and at 65° C. for 30 minutes in presence of 0.1×SSC and 0.1% SDS once; the following are the conditions for intermediate stringency: hybridization was done at 37° C. in buffer containing 25% formamide, 5×SSPE, 0.5% SDS and 50 ug/ml of sheared Herring DNA. The washing was performed at 50° C. for 30 minutes in presence of 1×SSC and 0.1% SDS once, at 50° C. for 30 minutes in presence of 0.5× SSC and 0.1% SDS once; the following are the conditions for low stringency: hybridization was done at 37° C. in buffer containing 25% formamide, 5×SSPE, 0.5% SDS and 50 ug/ml of sheared Herring DNA. The washing was performed at 37° C. for 30 minutes in presence of 1×SSC and 0.1% SDS once, at 37° C. for 30 minutes in presence of 0.5×SSC and 0.1% SDS once. A nucleic acid capable of hybridizing to a nucleic acid probe under conditions of high stringency will have about 80% to 100% identity to the probe; a nucleic acid capable of hybridizing to a nucleic acid probe under conditions of intermediate stringency will have about 50% to about 80% identity to the probe.

The term “hybridization” as used herein shall include “the process by which a strand of nucleic acid joins with a complementary strand through base pairing” (Coombs J (1994) Dictionary of Biotechnology, Stockton Press, New York N.Y.).

The process of amplification as carried out in polymerase chain reaction (PCR) technologies is described in Dieffenbach C W and G S Dveksler (1995, PCR Primer, a Laboratory Manual, Cold Spring Harbor Press, Plainview N.Y.). A nucleic acid sequence of at least about 10 nucleotides and as many as about 60 nucleotides from SEQ ID NO:1 preferably about 12 to 30 nucleotides, and more preferably about 25 nucleotides can be used as a probe or PCR primer.

A preferred method of isolating a nucleic acid construct of the invention from a cDNA or genomic library is by use of polymerase chain reaction (PCR) using oligonucleotide probes prepared on the basis of the polynucleotide sequence as shown in SEQ ID NO:1. For instance, the PCR may be carried out using the techniques described in U.S. Pat. No. 4,683,202.

Expression Systems

The present invention provides host cells, expression methods and systems for the production of phenol oxidizing enzymes obtainable from bacteria, yeast or non-Stachybotrys fungal species in host microorganisms. Such host microorganisms include fungus, yeast and bacterial species. Once nucleic acid encoding a phenol oxidizing enzyme of the present invention is obtained, recombinant host cells containing the nucleic acid may be constructed using techniques well known in the art. Molecular biology techniques are disclosed in Sambrook et al., Molecular Biology Cloning: A Laboratory Manual, Second Edition (1989) Cold Spring Harbor Laboratory Press, Cold Spring Harbor, N.Y. (1989). Nucleic acid encoding a phenol oxidizing enzyme of the present invention is obtained and transformed into a host cell using appropriate vectors. A variety of vectors and transformation and expression cassettes suitable for the cloning, transformation and expression in fungus, yeast and bacteria are known by those of skill in the art.

Typically, the vector or cassette contains sequences directing transcription and translation of the nucleic acid, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. These control regions may be derived from genes homologous or heterologous to the host as long as the control region selected is able to function in the host cell.

Initiation control regions or promoters, which are useful to drive expression of the phenol oxidizing enzymes in a host cell are known to those skilled in the art. Virtually any promoter capable of driving these phenol oxidizing enzyme is suitable for the present invention. Nucleic acid encoding the phenol oxidizing enzyme is linked operably through initiation codons to selected expression control regions for effective expression of the enzymes. Once suitable cassettes are constructed they are used to transform the host cell.

General transformation procedures are taught in Current Protocols In Molecular Biology (vol. 1, edited by Ausubel et al., John Wiley & Sons, Inc. 1987, Chapter 9) and include calcium phosphate methods, transformation using PEG and electroporation. For Aspergillus and Trichoderma, PEG and Calcium mediated protoplast transformation can be used (Finkelstein, D B 1992 Transformation. In Biotechnology of Filamentous Fungi. Technology and Products (eds by Finkelstein & Bill) 113-156. Electroporation of protoplast is disclosed in Finkelestein, D B 1992 Transformation. In Biotechnology of Filamentous Fungi. Technology and Products (eds by Finkelstein & Bill) 113-156. Microprojection bombardment on conidia is described in Fungaro et al. (1995) Transformation of Aspergillus nidulans by microprojection bombardment on intact conidia. FEMS Microbiology Letters 125 293-298. Agrobacterium mediated transformation is disclosed in Groot et al. (1998) Agrobacterium tumefaciens-mediated transformation of filamentous fungi. Nature Biotechnology 16 839-842. For transformation of Saccharomyces, lithium acetate mediated transformation and PEG and calcium mediated protoplast transformation as well as electroporation techniques are known by those of skill in the art.

Host cells which contain the coding sequence for a phenol oxidizing enzyme of the present invention and express the protein may be identified by a variety of procedures known to those of skill in the art. These procedures include, but are not limited to, DNA-DNA or DNA-RNA hybridization and protein bioassay or immunoassay techniques which include membrane-based, solution-based, or chip-based technologies for the detection and/or quantification of the nucleic acid or protein.

Phenol Oxidizing Enzyme Activities

The phenol oxidizing enzymes of the present invention are capable of using a wide variety of different phenolic compounds as electron donors, while being very specific for molecular oxygen as the electron acceptor and/or hydrogen peroxide as the electron acceptor.

Depending upon the specific substrate and reaction conditions, e.g., temperature, presence or absence of enhancers, etc., each phenol oxidizing enzyme oxidation reaction will have an optimum pH.

The phenol oxidizing enzymes of the present invention are capable of oxidizing a wide variety of dyes or colored compounds having different chemical structures, using oxygen and/or hydrogen peroxide as the electron acceptor. Accordingly phenol oxidizing enzymes of the present invention are used in applications where it is desirable to modify the color associated with dyes or colored compounds, such as in cleaning, for removing the food stains on fabric and anti-dye redeposition; textiles; and paper and pulp applications.

Colored Compounds

In the present invention, a variety of colored compounds could be targets for oxidation by phenol oxidizing enzymes of the present invention. For example, in detergent applications, colored substances which may occur as stains on fabrics can be a target. Several types or classes of colored substances may appear as stains, such as porphyrin derived structures, such as heme in blood stain or chlorophyll in plants; tannins and polyphenols (see P. Ribéreau-Gayon, Plant Phenolics, Ed. Oliver & Boyd, Edinburgh, 1972, pp.169-198) which occur in tea stains, wine stains, banana stains, peach stains; carotenoids, the coloured substances which occur in tomato (lycopene, red), mango (carotene, orange-yellow) (G. E. Bartley et al., The Plant Cell (1995), Vol 7, 1027-1038); anthocyanins, the highly colored molecules which occur in many fruits and flowers (P. Ribéreau-Gayon, Plant Phenolics, Ed. Oliver & Boyd, Edinburgh, 1972, 135-169); and Maillard reaction products, the yellow/brown colored substances which appear upon heating of mixtures of carbohydrate molecules in the presence of protein/peptide structures, such as found in cooking oil. Pigments are disclosed in Kirk-Othmer, Encyclopedia of Chemical Technology, Third edition Vol. 17; page 788-889, a Wiley-Interscience publication. John Wiley & Sons and dyes are disclosed in Kirk-Othmer, Encyclopedia of Chemical Technology, Third edition, vol. 8, a Wiley-interscience publication. John Wiley & Sons.

Enhancers

A phenol oxidizing enzyme of the present invention may act to modify the color associated with dyes or colored compounds in the presence or absence of enhancers depending upon the characteristics of the compound. If a compound is able to act as a direct substrate for the phenol oxidizing enzyme, the phenol oxidizing enzyme can modify the color associated with a dye or colored compound in the absence of an enhancer, although an enhancer may still be preferred for optimum phenol oxidizing enzyme activity. For other colored compounds unable to act as a direct substrate for the phenol oxidizing enzyme or not directly accessible to the phenol oxidizing enzyme, an enhancer is required for optimum phenol oxidizing enzyme activity and modification of the color.

Enhancers are described in for example WO 95/01426 published 12 Jan. 1995; WO 96/06930, published 7 Mar. 1996; and WO 97/11217 published 27 Mar. 1997. Enhancers include but are not limited to phenothiazine-10-propionic acid (PPT), 10-methylphenothiazine (MPT), phenoxazine-10-propionic acid (PPO), 10-methylphenoxazine (MPO), 10-ethylphenothiazine-4-carboxylic acid (EPC) acetosyringone, syringaldehyde, methylsyringate, 2,2′-azino-bis (3-ethylbenzothiazoline-6-sulfonate (ABTS) and 4-Hydroxy-4-biphenyl-carboxylic acid.

Cultures

The present invention encompasses phenol oxidizing enzymes obtainable from fungus including but not limited to Myrothecium species, Curvalaria species, Chaetomium species, Bipolaris species, Humicola species, Pleurotus species, Trichoderma species, Mycellophthora species and Amerosporium species. In particular, the fungus includes but is not limited to Myrothecium verrucaria, Curvalaria pallescens, Chaetomium sp, Bipolaris spicifera, Humicola insolens, Pleurotus abalonus, Trichoderma reesei, Mycellophthora thermophila and Amerosporium atrum. In addition to the illustrative examples provided herein, other examples of the above species include Myrothecium verrucaria having ATCC accession number 36315; Pleurotus abalonus having ATCC accession number 96053; Humicola insolens having ATCC accession number 22082; Mycellophth ora thermophila having ATCC accession number 48104; and Trichoderma reesei having ATCC Accession Number 56765.

Purification

The phenol oxidizing enzymes of the present invention may be produced by cultivation of phenol oxidizing enzyme-producing strains under aerobic conditions in nutrient medium containing assimiable carbon and nitrogen together with other essential nutrient(s). The medium can be composed in accordance with principles well-known in the art.

During cultivation, the phenol oxidizing enzyme-producing strains secrete phenol oxidizing enzyme extracellularly. This permits the isolation and purification (recovery) of the phenol oxidizing enzyme to be achieved by, for example, separation of cell mass from a culture broth (e.g. by filtration or centrifugation). The resulting cell-free culture broth can be used as such or, if desired, may first be concentrated (e.g. by evaporation or ultrafiltration). If desired, the phenol oxidizing enzyme can then be separated from the cell-free broth and purified to the desired degree by conventional methods, e.g. by column chromatography, or even crystallized.

The phenol oxidizing enzymes of the present invention may be isolated and purified from the culture broth into which they are extracellularly secreted by concentration of the supernatant of the host culture, followed by ammonium sulfate fractionation and gel permeation chromatography. As described herein in Example I for Stachybotrys chartarum phenol oxidizing enzyme, the phenol oxidizing enzymes of the present invention may be purified and subjected to standard techniques for protein sequencing. Oligonucleotide primers can be designed based on the protein sequence and used in PCR to isolate the nucleic acid encoding the phenol oxidizing enzyme. The isolated nucleic acid can be characterized and introduced into host cells for expression. Accordingly, the present invention encompasses expression vectors and recombinant host cells comprising a phenol oxidizing enzyme of the present invention and the subsequent purification of the phenol oxidizing enzyme from the recombinant host cell.

The phenol oxidizing enzymes of the present invention may be formulated and utilized according to their intended application. In this respect, if being used in a detergent composition, the phenol oxidizing enzyme may be formulated, directly from the fermentation broth, as a coated solid using the procedure described in U.S. Pat. No. 4,689,297. Furthermore, if desired, the phenol oxidizing enzyme may be formulated in a liquid form with a suitable carrier. The phenol oxidizing enzyme may also be immobilized, if desired.

Assays for Phenol Oxidizing Activity

Phenol oxidizing enzymes can be assayed for example by ABTS activity as described in Example II or by the delignification method as disclosed in Example III or in detergent methods known by those of skill in the art.

Detergent Compositions

A phenol oxidizing enzyme of the present invention may be used in detergent or cleaning compositions. Such compositions may comprise, in addition to the phenol oxidizing enzyme, conventional detergent ingredients such as surfactants, builders and further enzymes such as, for example, proteases, amylases, lipases, cutinases, cellulases or peroxidases. Other ingredients include enhancers, stabilizing agents, bactericides, optical brighteners and perfumes. The detergent compositions may take any suitable physical form, such as a powder, an aqueous or non aqueous liquid, a paste or a gel. Examples of detergent compositions are given in WO 95/01426, published 12 Jan. 1995 and WO 96/06930 published 7 Mar. 1996.

Having thus described the phenol oxidizing enzymes of the present invention, the following examples are now presented for the purposes of illustration and are neither meant to be, nor should they be, read as being restrictive. Dilutions, quantities, etc. which are expressed herein in terms of percentages are, unless otherwise specified, percentages given in terms of per cent weight per volume (w/v). As used herein, dilutions, quantities, etc., which are expressed in terms of % (v/v), refer to percentage in terms of volume per volume. Temperatures referred to herein are given in degrees centigrade (C). All patents and publications referred to herein are hereby incorporated by reference.

### EXAMPLE I

Stachybotrys chartarum phenol oxidizing enzyme production Stachybotrys chartarum ATCC accession number 38898 was grown on PDA plates (Difco) for about 5-10 days. A portion of the plate culture (about ¾×¾ inch) was used to inoculate 100 ml of PDB (potato dextrose broth) in 500-ml shake flask. The flask was incubated at 26-28 degrees C., 150 rpm, for 3-5 days until good growth was obtained.

The broth culture was then inoculated into 1 L of PDB in a 2.8-L shake flask. The flask was incubated at 26-28 degrees C., 150 rpm, for 2-4 days until good growth was obtained.

A 10-L fermentor containing a production medium was prepared (containing in grams/liter the following components: glucose 15; lecithin 1.51; t-aconitic acid 1.73; KH2PO4 3; MgSO4.7H2O 0.8; CaCl2.2H2O 0.1; ammonium tartrate 1.2; soy peptone 5; Staley 7359; benzyl alcohol 1; tween 20 1; nitrilotriacetic acid 0.15; MnSO4.7H2O 0.05; NaCl 0.1; FeSO4.7H2O 0.01; CoSO4 0.01; CaCl2.2H2O 0.01; ZnSO4.7H2O 0.01; CuSO4 0.001; ALK(SO4)2.12H2O 0.001; H3BO3 0.001; NaMoO4.2H2O 0.001). The fermentor was then inoculated with the 1-L broth culture, and fermentation was conducted at 28 degrees C. for 60 hours, under a constant air flow of 5.0 liters/minute and a constant agitation of 120 RPM. The pH was maintained at 6.0.

The presence of phenol oxidizing enzyme activity in the supernatant was measured using the following assay procedure, based on the oxidation of ABTS (2,2′-azino-bis-(3-ethylbenzothiazoline-6-sulfonate)) by oxygen. ABTS (SIGMA, 0.2 ml, 4.5 mM H2O) and NaOAc (1.5 ml, 120 mM in H2O, pH 5.0) were mixed in a cuvette. The reaction was started by addition of an appropriate amount of the preparation to be measured (which in this example is the supernatant dilution) to form a final solution of 1.8 ml. The color produced by the oxidation of ABTS was then measured every 2 seconds for total period of 14 seconds by recording the optical density (OD) at 420 nm, using a spectrophotometer. One ABTS unit (one enzyme unit or EACU) in this example is defined as the change in OD measured at 420 per minute/2 (given no dilution to the sample). In this manner a phenol oxidizing enzyme activity of 3.5 EACU/ml of culture supernatant was measured.

The resulting supernatant was then removed from the pellet and concentrated to 0.6 liters by ultrafiltration using a Amicon ultrafiltration unit equipped with a YMIO membrane having a 10 kD cutoff.

A volume of 1.4 liters of acetone was added to the concentrate and mixed therewith. The resulting mixture was then incubated for two hours at 20-25 degrees C.

Following incubation, the mixture was centrifuged for 30 minutes at 10,000 g and the resulting pellet was removed from the supernatant. The pellet was then resuspended in a final volume of 800 ml of water.

The resulting suspension was then submitted to ammonium sulfate fractionation as follows: crystalline ammonium sulfate was added to the suspension to 40% saturation and the mixture incubated at 4 degrees C. for 16 hours with gentle magnetic stirring. The mixture was then centrifuged at 10,000 g for 30 minutes and the supernatant removed from the centrifugation pellet for further use. Ammonium sulfate was then added to the supernatant to reach 80% saturation, and the mixture incubated at 4 degrees C. for 16 hours with gentle magnetic stirring. The suspension was then centrifuged for 30 minutes at 10,000 g and the resulting pellet was removed from the supernatant. The pellet was then resuspended in 15 ml of water and concentrated to 6 ml by ultrafiltration using a CENTRIPREP 3000 (AMICON).

The phenol oxidizing enzyme activity of the suspension was then measured using the standard assay procedure, based on the oxidation of ABTS by oxygen, as was described above (but with the exception that the preparation being assayed is the resuspended concentration and not the supernatant dilutions). The phenol oxidizing enzyme activity so measured was 5200 EU/ml.

The enzyme was then further purified by gel permeation chromatography. In this regard, a column containing 850 ml of SEPHACRYL S400 HIGH RESOLUTION (PHARMACIA) was equilibrated with a buffer containing 50 mM KH2PO4/K2HPO4 (pH=7.0) and then loaded with the remainder of the 6 ml suspension described above, and eluted with the buffer containing 50 mM KH2PO4/K2HPO4 (pH=7.0), at a flow rate of 1 ml/minute. Respective fractions were then obtained.

The respective fractions containing the highest phenol oxidizing enzyme activities were pooled together, providing a 60 ml suspension containing the purified phenol oxidizing enzyme.

The phenol oxidizing enzyme activity of the suspension was then measured based on the oxidation of ABTS by oxygen. The enzyme activity so measured was 390 EU/ml. Stachybotrys chartarum phenol oxidizing enzyme prepared as disclosed above was subjected to SDS polyacrylamide gel electrophoresis and isolated. The isolated fraction was treated with urea and iodoacetamide and digested by the enzyme endoLysC. The fragments resulting from the endoLysC digestion were separated via HPLC (reverse phase monobore C18 column, CH3CN gradient) and collected in a multititer plate. The fractions were analysed by MALDI for mass determination and sequenced via Edman degradation. The following amino acid sequences were determined and are shown in amino terminus to carboxy terminus orientation:

N′ DYYFPNYQSARLLXYHDHA C′
N′ RGQVMPYESAGLK C′

Two degenerated primers were designed based on the peptide sequence. Primer 1 contains the following sequence: TATTACTTTCCNAAYTAYCA where N represents a mixture of all four nucleotides (A, T, C and G) and Y represents a mixture of T and C only. Primer 2 contains the following sequence:

TCGTATGGCATNACCTGNCC.

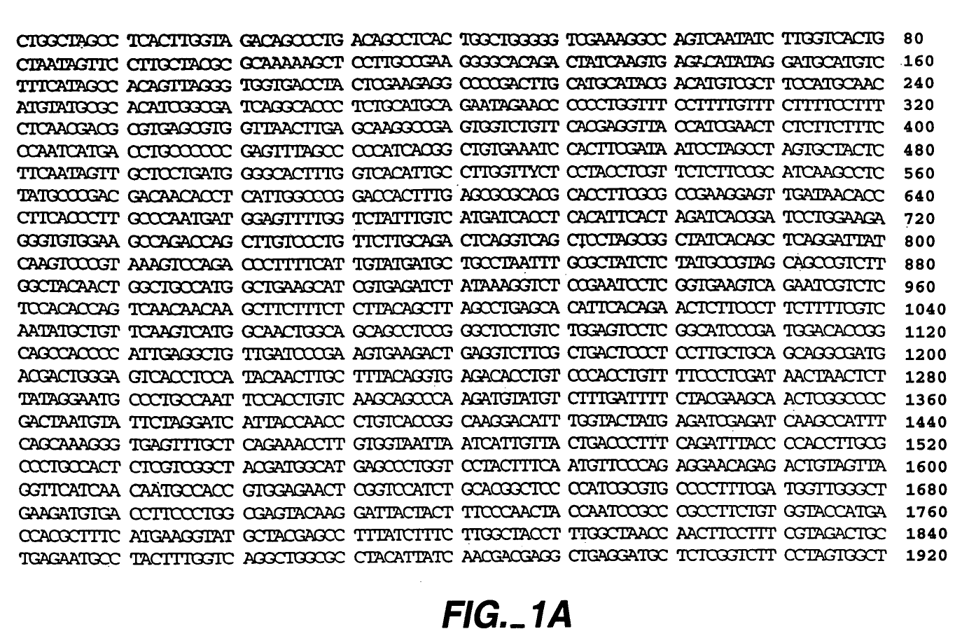

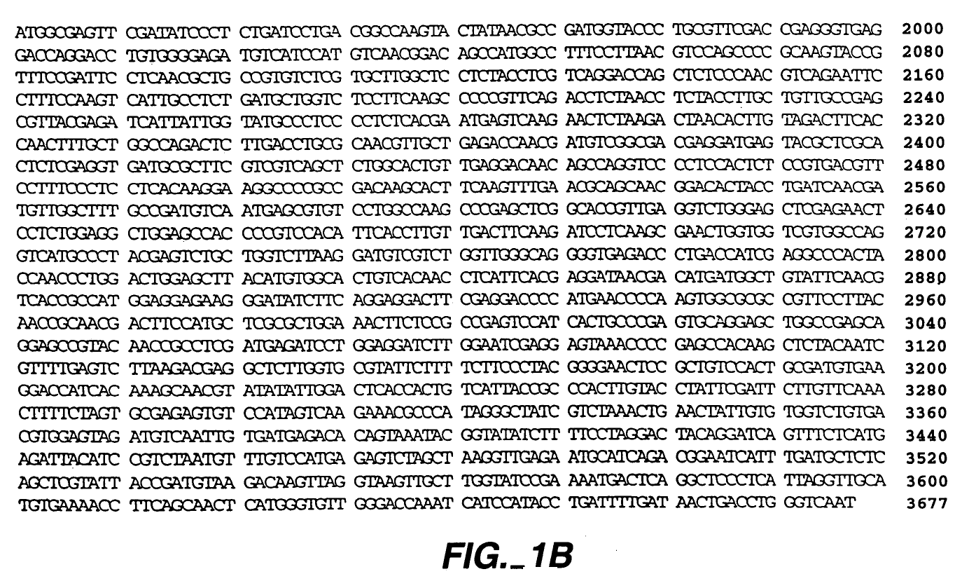

For isolation of genomic DNA encoding phenol oxidizing enzyme, DNA isolated from Stachybotrys chartarum (MUCL # 38898) was used as a template for PCR. The DNA was diluted 100 fold with Tris-EDTA buffer to a final concentration of 88 ng/ul. Ten microliter of diluted DNA was added to the reaction mixture which contained 0.2 mM of each nucleotide (A, G. C and T), 1× reaction buffer, 0.296 microgram of primer 1 and 0.311 microgram of primer 2 in a total of 100 microliter reaction. After heating the mixture at 100° C. for 5 minutes, 2.5 units of Taq DNA polymerase was added to the reaction mix. The PCR reaction was performed at 95° C. for 1 minute, the primers were annealed to the template at 45° C. for 1 minute and extension was done at 68° C. for 1 minute. This cycle was repeated 30 times to achieve a gel-visible PCR fragment. The PCR fragment detected by agarose gel contained a fragment of about 1 kilobase which was then cloned into the plasmid vector pCR-II (Invitrogen). The 1 kb insert was then subjected to nucleic acid sequencing. The sequence data revealed that it was the gene encoding Stachybotrys chartarum because the deduced peptide sequence matched the peptide sequences disclosed above sequenced via Edman degradation. The PCR fragments containing the 5′ gene and 3′ gene were then isolated and sequenced. FIG. 1 provides the full length genomic sequence (SEQ ID NO:1) of Stachybotrys oxidase including the promoter and terminator sequences.

### EXAMPLE II

The following example describes the ABTS assay used for the determination of phenol oxidizing activity. The ABTS assay is a spectrophotometric activity assay which uses the following reagents: assay buffer=50 sodium acetate, pH 5.0; 50 mM sodium phosphate, pH 7.0; 50 mM sodium carbonate, pH 9.0. The ABTS (2,2′-azinobis 3 ethylbenzothiazoline-6-sulphonic acid]) is a 4.5 mM solution in distilled water.

0.75 ml assay buffer and 0.1 ml ABTS substrate solution are combined, mixed and added to a cuvette. A cuvette containing buffer-ABTS solution is used as a blank control. 0.05 ml of enzyme sample is added, rapidly mixed and placed into the cuvette containing buffer-ABTS solution. The rate of change in absorbance at 420 nm is measure, DOD 420/minute, for 15 seconds (or longer for samples having activity rates<0.1) at 30° C. Enzyme samples having a high rate of activity are diluted with assay buffer to a level between 0.1 and 1.

### EXAMPLE III

This example a shake flask pulp bleaching protocol used to determine the activity of phenol oxidizing enzymes.

The buffer used is 50 mM Na Acetate, pH 5 or 50 mM Tris pH 8.5. Softwood oxygen delignified pulp with a of kappa 17.3 is used. The enzyme is dosed at 10 ABTS units per g of pulp. The assay can be performed with and without mediators, such as those described infra.

250 ml of pre-warmed buffer is placed in a graduated cylinder. 10 g of wet pulp (at 72% moisture=2.8 g dry pulp) is placed into a standard kitchen blender with ˜120 ml buffer. The pulp is blended on the highest setting for about 30 seconds. The resulting slurry is placed into a large-mouth shake flask (residual pulp is rinsed out of the blender with remaining buffer and spatula) which results in about a 1% consistency in the flask (2.8 g/250 ml).

The enzyme +/− mediator is added and controls without enzyme are included in the assay. The opening of the flask is covered with 2 thickness cheese cloth and secured with a rubber band. The flasks are placed into a shaker and incubated for 2 hours at ˜55° C. and 350 rpm.

At the end of the incubation time, 500 mls of 2% NaOH are added directly into the flasks and the shaker temperature is set to 70° C. and allowed to incubate for 1.5 hours at 250 rpm. The flask contents are filtered through buchner funnels. The pulp slurries are poured directly into the funnels, without vacuum and are allowed to slowly drip which sets up a filter layer inside the funnel.

Once most of the flask contents are in the funnel, a light vacuum is applied to pull the material into a cake inside the funnel. The filtrate (liquid) is poured back into the original shake flask and swirled to wash residual pulp from the sides. The filtrate is poured back on top of the filter cake. The end result is a fairly clear light golden colored filtrate with most of the pulp caught in the funnel. The filter cake is washed without vacuum, by gently pouring 1 liter of DI water over the filter cake and letting it drip through on its own. A vacuum is applied only at the end to suck the cake dry. The filter cakes are dried in the funnels overnight in a 100° C. oven. The dried pulp is manually scraped from the cooled funnels the next day. Microkappa determinations based on the method of the Scandinavian Pulp, Paper and Board Testing committee Scan-c 1:77 (The Scandinavian Pulp, Paper and Board Testing committee Box 5604,S-114, 86 Stockholm, Sweden) are performed to determine % delignification.

### EXAMPLE IV

Example IV describes the Southern hybridization technique used to identify homologous genes from other organisms

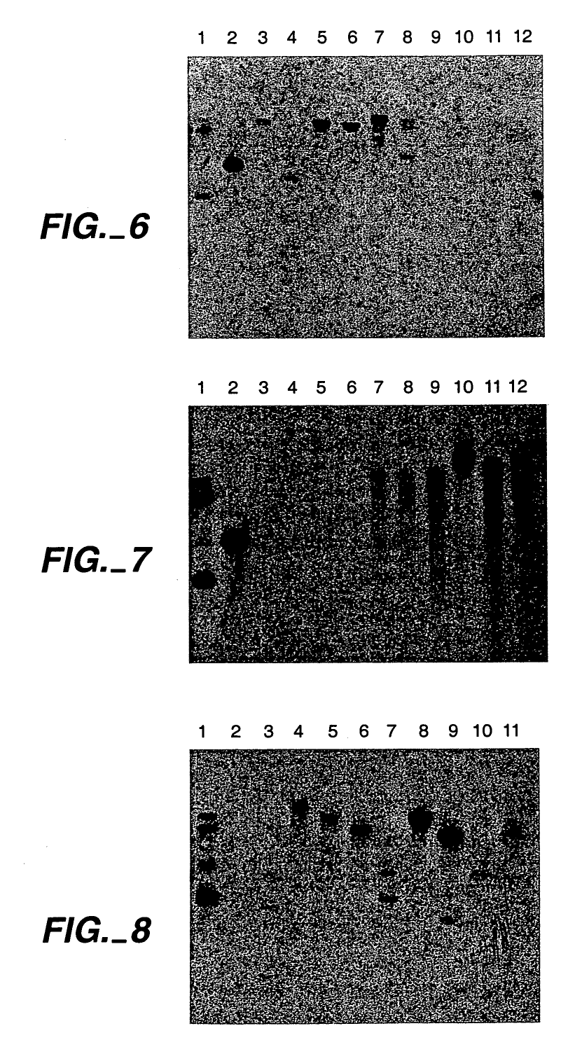

The genomic DNA from several fungal strains including the Stachybotrys chartarum, Myrothecium verruvaria, Myrothecium cinctum, Curvalaria pallescens, Humicola insulas, Pleurotus eryngii, Pleurotus abalous, Aspergillus niger, Corpinus cineras, Mycellophthora thermophila, Trichoderma reesei, Trametes vesicolor, Chaetomium sp, and Bipolaris spicifera was isolated. All fungal species were grown in either CSL medium (described in Dunn-Coleman et al., 1991, Bio/Technology 9:976-981) or MB medium (glucose 40 g/l; soytone 10 g/l; MB trace elements 1 ml/L at pH 5.0) for 2 to 4 days. The mycelia were harvested by filtering through Mirocloth (Calbiochem). The genomic DNA was extracted from cells by repeated phenol/chloroform extraction according to the fungal genomic DNA purification protocol (Hynes M J, Corrick C M, King J A 1983, Mol Cell Biol 3:1430-1439). Five micrograms genomic DNA were digested with restriction enzyme EcoRI or Hind III overnight at 37° C. and the DNA fragments were separated on 1% agarose gel by electrophoresis in TBE buffer. The DNA fragments were then transferred from agarose gel to the Nitrocellulose membrane in 20×SSC buffer. The probe used for Southern analysis was isolated from plasmids containing either the entire coding region of the Stachybotrys phenol oxidizing enzyme (SEQ ID NO:1) or a DNA fragment generated through PCR reaction that covers the internal part of the genes of more than 1 kb in size. The primers used to generate the PCR fragment were Primer 1 containing the following sequence: TATTACTTTCCNAAYTAYCA where N represents a mixture of all four nucleotides (A, T, C and G) and Y represents a mixture of T and C only and Primer 2 containing the following sequence: TCGTATGGCATNACCTGNCC. Southern hybridizations were performed for 18 to 20 hours at 37° C. in an intermediate stringency hybridization buffer containing 25% formamide, 5×SSPE, 0.5% SDS and 50 ug/ml of sheared Herring DNA. The blots were washed once at 50° C. for 30 minutes in presence of 1×SSC and 0.1% SDS and washed again at 50° C. for 30 minutes in 0.5×SSC and 0.1% SDS. The Southern blots were exposed to x-ray film for more than 20 hours and up to 3 days. FIGS. 6, 7,and 8 showed that the genomic DNAs of several fungal species contained sequences that were able to hybridize under the conditions described above to the nucleic acid encoding the Stachybotrys phenol oxidizing enzyme shown in SEQ ID NO:1. These fungal species giving the strongest signal (which may indicate a higher identity to the nucleic acid probe than those giving a weaker signal) are Myrothecium verrucaria, Curvalaria pallescens, Chaetomium sp, Bipolaris spicifera, and Amerosporium atrum. Fungal species also hybridizing to nucleic acid encoding the Stachybotrys phenol oxidizing enzyme were detected from genomic DNA of Humicola insolens, Pleurotus abalonus, Trichoderma reesei and Mycellophthora thermophila.

### EXAMPLE V

Example V describes the cloning of genes encoding fungal enzymes capable of hybridizing to Stachybotrys phenol oxidizing enzyme of SEQ ID NO:1.

A. Bipolaris spicifera

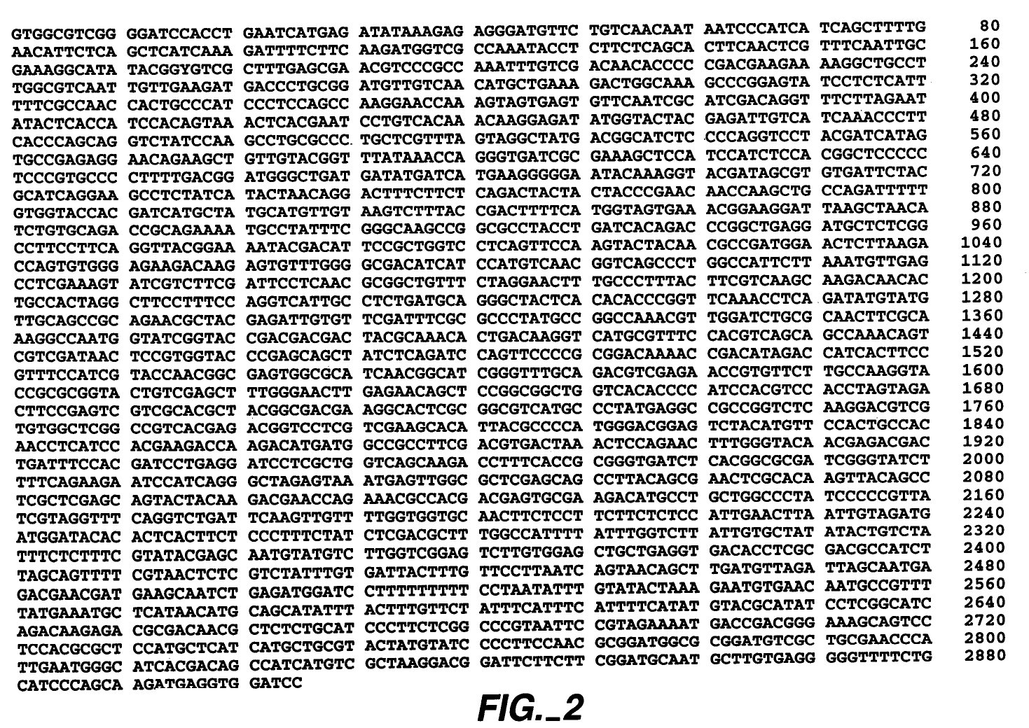

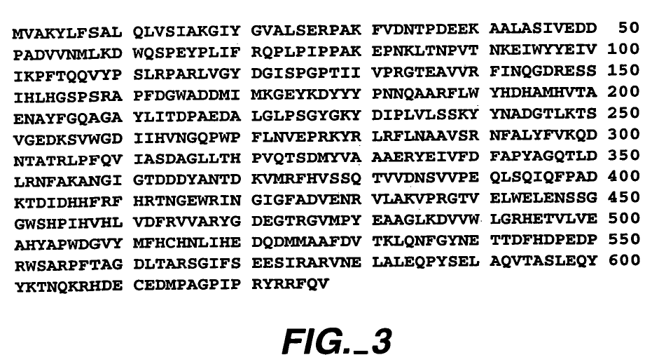

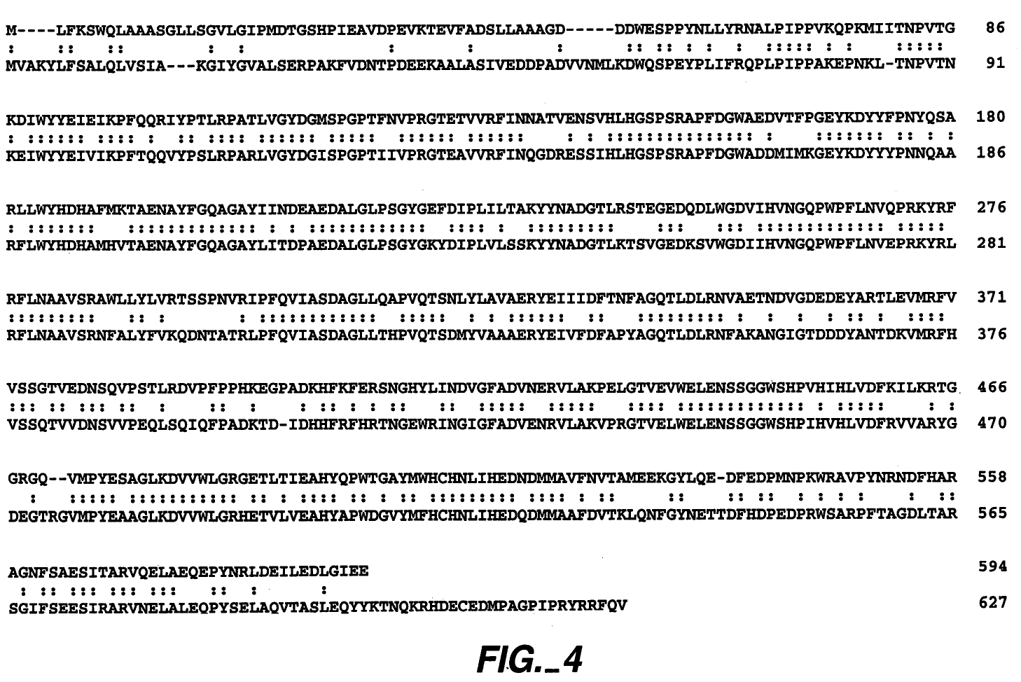

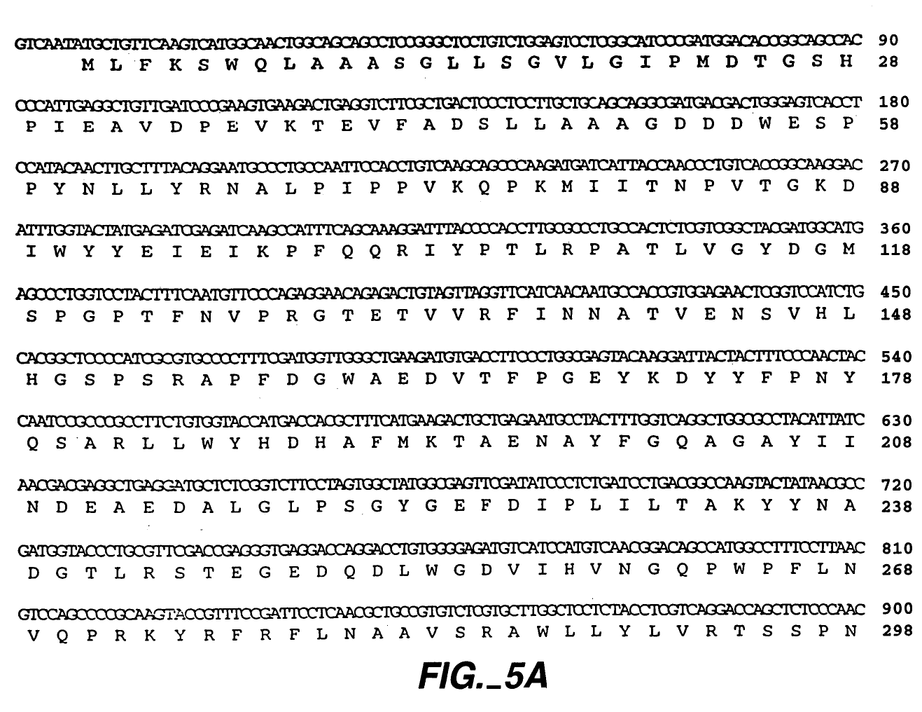

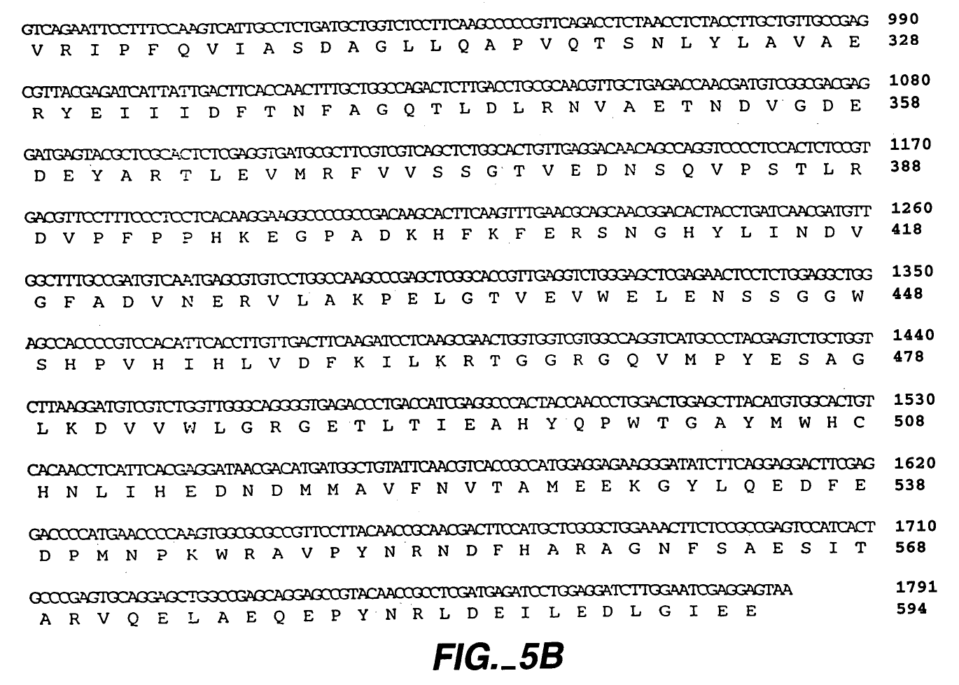

Based on the DNA and protein sequences comparison of the phenol oxidizing enzyme of SEQ ID NO:1 (from the Stachybotrys chartarum) and bilirubin oxidase from the Myrothecium verruvaria (GenBank number 14081), a set of oligonucleotide primers was designed to isolate related sequences from a number of different organisms via hybridization techniques. The following oligonucleotide primers (primer 1: 5′ TGGTACCAYGAYCAYGCT 3′ and primer 2: 5′ RGACTCGTAKGGCATGAC 3′ (where the Y is an equal mixture of nucleotides T and C, R is an equal mixture of nucleotides A and G and K represents an equal mixture of nucleotides T and G) were used to clone a phenol oxidizing enzyme from Bipolaris spicifera. The genomic DNA isolated from Bipolaris spicifera was diluted 10 fold with Tris-EDTA buffer to a final concentration of 63 ng/ul. Ten microliters of diluted DNA were added to a reaction mixture which contained 0.2 mM of each nucleotide (A, G. C and T), 1× reaction buffer (10mM Tris, 1.5 mM MgCl2, 50 mM KCl at pH 8.3) in a total of 100 microliters reaction in the presence of primers 1 and 2. After heating the mixture at 100° C. for 5 minutes, 2.5 units of Taq DNA polymerase was added to the reaction mix. The PCR reaction was performed at 95° C. for 1 minute, the primer was annealed to the template at 50° C. for 1 minute and extension was done at 72° C. for 1 minute. This cycle was repeated 30 times to achieve a gel-visible PCR fragment and an extension at 72° C. for 7 minutes was added after 30 cycles. The PCR fragment detected by agarose gel contained a fragment of about 1 kilobase which was then cloned into the plasmid vector pCR-II (Invitrogen). The 1 kb insert was then subjected to nucleic acid sequencing. The 3′ end of the gene was isolated by RS-PCR method (Sarkar et al., 1993, PCR Methods and Applications 2:318-322) from the genomic DNA of the Bipolaris spicifera. The PCR fragment was cloned into the plasmid vector pCR-II (Invitrogen) and sequenced. The 5′ end of the gene was isolated by the same RS-PCR method (Sarkar et al 1993, PCR methods and applications 2:318-322) from the genomic DNA of the Bipolaris spicifera. The PCR fragment was also cloned into the plasmid vector pCR-II (Invitrogen) and sequenced. The full length genomic DNA (SEQ ID NO:3) including the regulatory sequence of the promoter and terminator regions is shown in FIG. 2 and the amino acid sequence translated from genomic DNA is shown in FIG. 3 (SEQ ID NO:4). The sequence data comparison, shown in FIG. 4, revealed that it encodes a phenol oxidizing enzyme having about 60.8% identity to the Stachybotrys chartarum phenol oxidizing enzyme shown in SEQ ID NO:1 (as determined by MegAlign Program from DNAstar (DNASTAR, Inc. Maidson, Wis. 53715) by Jotun Hein Method (1990, Method in Enzymology, 183: 626-645) with a gap penalty=11, a gap length penalty=3 and Pairwise Alignment Parameters Ktuple=2.

B. Curvularia pallescens

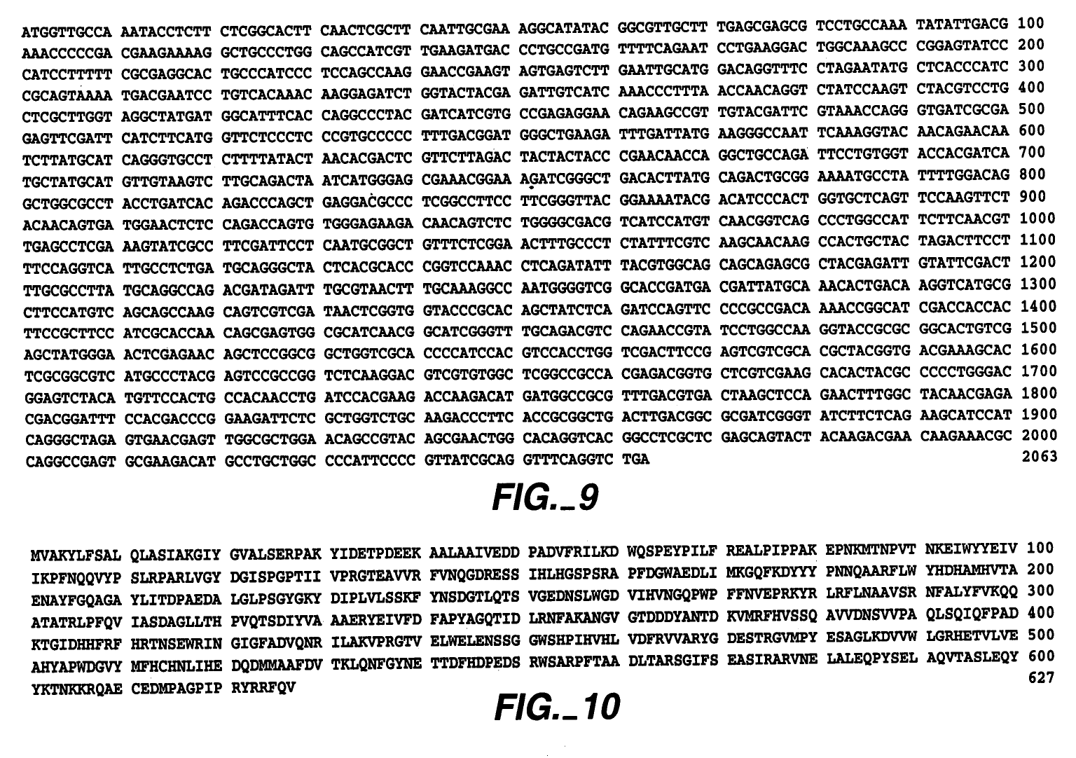

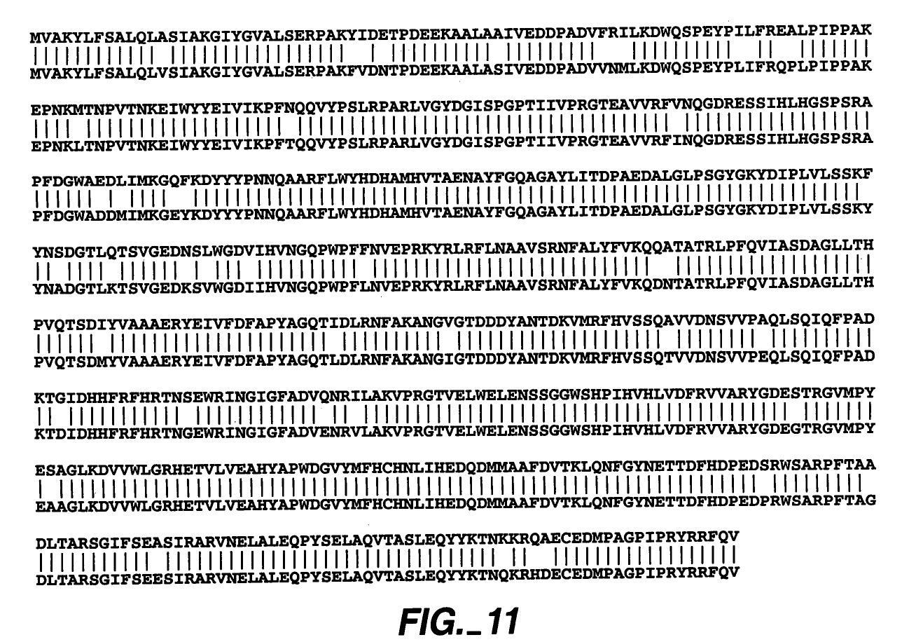

Based on the comparison of the nucleic acid and protein sequences of the phenol oxidizing enzyme of SEQ ID NO:1 (obtainable from Stachybotrys chartarum) and bilirubin oxidase obtainable from Myrothecium verruvaria (GenBank accession number 14081), a set of oligonucleotide primers was designed to isolate related sequences from a number of different organisms via hybridization techniques. The following oligonucleotide primers (primer 1: 5′ TGGTACCAYGAYCAYGCT 3′ and primer 2: 5′ TCGTGGATGARRTTGTGRCAR 3′ (where the Y is an equal mixture of nucleotides T and C, R is an equal mixture of nucleotides A and G) were used to clone a phenol oxidizing enzyme from Curvularia pallescens. The genomic DNA isolated from Curvularia pallescens was diluted with Tris-EDTA buffer to a final concentration of 200 ng/ul. Ten microliters of diluted DNA were added to a reaction mixture which contained 0.2 mM of each nucleotide (A, G. C and T), 1× reaction buffer (10 mM Tris, 1.5 mM MgCl2, 50 mM KCl at pH 8.3) in a total of 100 microliters reaction in the presence of primers 1 and 2. After heating the mixture at 100° C. for 5 minutes, 2.5 units of Taq DNA polymerase were added to the reaction mix. The PCR reaction was performed at 95° C. for 1 minute, the primer was annealed to the template at 50° C. for 1 minute and extension was done at 72° C. for 1 minute. This cycle was repeated 30 times and an extension at 72° C. for 7 minutes was added after 30 cycles. The PCR fragment detected by agarose gel contained a fragment of about 900 base pairs. The 900 bp PCR fragment was then subjected to nucleic acid sequencing. The 5′ and part of 3′ end of the genomic DNA was isolated by inverse PCR method (Triglia T et al, Nucleic Acids Res. 16:8186) from the genomic DNA of Curvularia pallescens using two pairs of oligonucleotides based on sequence data from the 900 bp PCR fragment. The full length genomic DNA (SEQ ID NO:6) from the translation start site to the translation stop site is shown in FIG. 9 and the putative amino acid sequence translated from genomic DNA is shown in FIG. 10 (SEQ ID NO:7). The sequence data comparison, shown in FIG. 11, illustrates that the phenol oxidizing enzyme obtainable from Curvularia pallescens and having SEQ ID NO:7 has 92.8% identity to the phenol oxidizing enzyme cloned from Bipolaris spicifera shown in SEQ ID NO:4 (as determined by MegAlign Program from DNAstar (DNASTAR, Inc. Maidson, Wis. 53715) by Jotun Hein Method (1990, Method in Enzymology, 183: 626-645) with a gap penalty=11, a gap length penalty=3 and Pairwise Alignment Parameters Ktuple=2. SEQ ID NO:7 has 60.8% identity to the Stachybotrys oxidase phenol oxidizing enzyme A shown in SEQ ID NO:1.

C. Amerosporium atrum

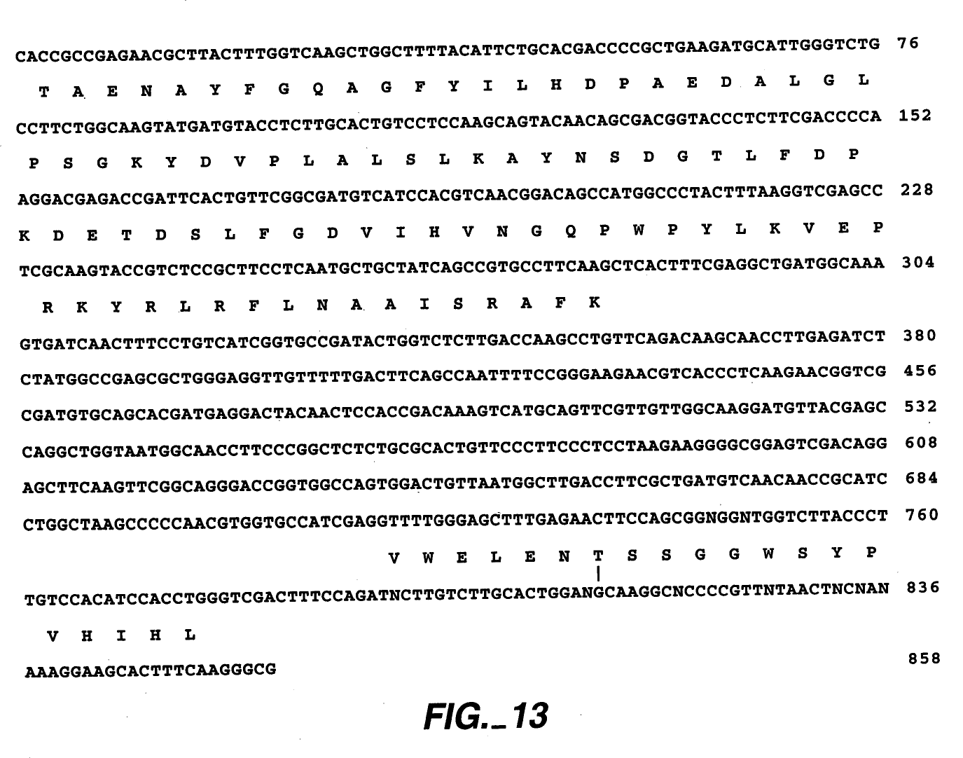

Based on the DNA and protein sequences comparison of the phenol oxidizing enzyme of SEQ ID NO:1 (from the Stachybotrys chartarum) and bilirubin oxidase from the Myrothecium verruvaria (GenBank number 14081), a set of oligonucleotide primers was designed to isolate related sequences from a number of different organisms via hybridization techniques. The following oligonucleotide primers (primer 1: 5′ TGGTACCAYGAYCAYGCT 3′ and primer 2: 5′ CXAGACRACRTCYTTRAGACC 3′ (where the Y is an equal mixture of nucleotides T and C, R is an equal mixture of nucleotides A and G and X is an equal mixture of nucleotides G, A, T and C) were used to clone a phenol oxidizing enzyme from Amerosporium atrum . A reaction mixture which contained 0.2 mM of each nucleotide (A, G. C and T), 1× reaction buffer (10 mM Tris, 1.5 mM MgCl/2, 50 mM KCl at pH 8.3), 1 ul of 50 pmol/ul primers 1 and 2 in a total of 50 microliters reaction were added to a hot start tube (Molecular Bio-Products). The mixture was heated to 95 C for 90 seconds, and the tubes were cooled on ice for 5 minutes. The genomic DNA isolated from Amerosporium atrum was diluted 10 fold with Tris-EDTA buffer to a final concentration of 41 ng/ul. About 1 ul of the diluted DNA was added to the hot start tube with 1× reaction buffer (10 mM Tris, 1.5 mM MgCl2, 50 mM KCl at pH 8.3), 2.5 units of Taq DNA polymerase in a total volume to 50 microliters. The reaction mixture was heated to 95 C for 5 minutes. The PCR reaction was performed at 95° C. for 1 minute, the primer was annealed to the template at 51° C. for 1 minute and extension was done at 72° C. for 1 minute. This cycle was repeated 29 times to achieve a gel-visible PCR fragment and an extension at 72° C. for 7 minutes was added after 29 cycles. The PCR fragment detected by agarose gel contained a fragment of about 1 kilobase. The 1 kb insert was then subjected to nucleic acid sequencing. The genomic sequence for the Amerosporium atrum is shown in FIG. 13. An amino acid alignment of the amino acid obtainable from Amerosporium atrum and SEQ ID NO:2 is shown in FIG. 14.

### EXAMPLE VI

Example VI illustrates the Bipolaris spicifera pH profile as measured at 470 nm using Guaicol as a substrate.

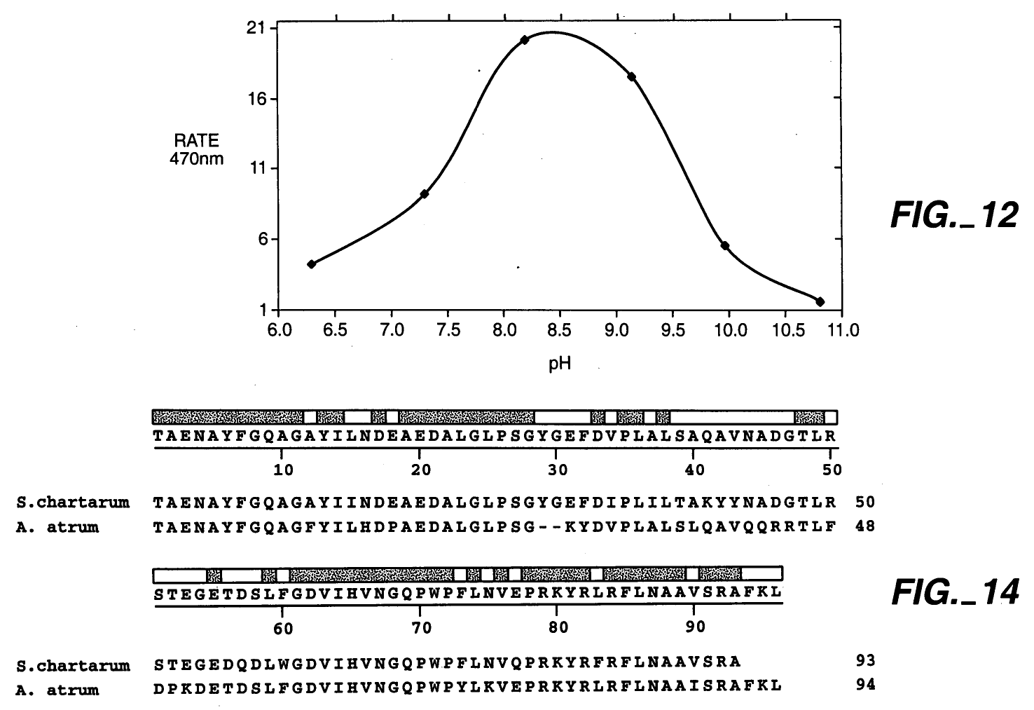

Phenol oxidizing enzyme obtainable from Bipolaris spicifera was diluted in water and added to 96 well plates which contained the Briton and Robinson buffer system at a final concentration of 20 mM. Guaicol (Sigma catalog number 6-5502) was added to the wells at a final concentration of 1 mM. The reaction was allowed to proceed for 15′ at a temperature of 25° C. and a reading was taken every 11 minutes using a spectrophotometer at a lambda of 470 nm. The results are shown in FIG. 12.

The Briton and Robinson buffer system is shown in Table 1 below.

TABLE I
x mL of 0.2 M NaOH Added to 100 mL of Stock Solution
(0.04 M Acetic Acid, 0.04 M H₃PO₄, and 0.04 M Boric Acid)

| pH | NaOH, mL |
|---|---|
| 1.81 | 0.0 |
| 1.89 | 2.5 |
| 1.98 | 5.0 |
| 2.09 | 7.5 |
| 2.21 | 10.0 |
| 2.36 | 12.5 |
| 2.56 | 15.0 |
| 2.87 | 17.5 |
| 3.29 | 20.0 |
| 3.78 | 22.5 |
| 4.10 | 25.0 |
| 4.35 | 27.5 |
| 4.56 | 30.0 |
| 4.78 | 32.5 |
| 5.02 | 35.0 |
| 5.33 | 37.5 |
| 5.72 | 40.0 |
| 6.09 | 42.5 |
| 6.37 | 45.0 |
| 6.59 | 47.5 |
| 6.80 | 50.0 |
| 7.00 | 52.5 |
| 7.24 | 55.0 |
| 7.54 | 57.5 |
| 7.96 | 60.0 |
| 8.36 | 62.5 |
| 8.69 | 65.0 |
| 8.95 | 67.5 |
| 9.15 | 70.0 |
| 9.37 | 72.5 |
| 9.62 | 75.0 |
| 9.91 | 77.5 |
| 10.38 | 80.0 |
| 10.88 | 82.5 |
| 11.20 | 85.0 |
| 11.40 | 87.5 |
| 11.58 | 90.0 |
| 11.70 | 92.5 |
| 11.82 | 95.0 |
| 11.92 | 97.5 |

### EXAMPLE VII

Example VII illustrates the bleaching of tomato stains by phenol oxidizing enzyme obtainable from Bipolaris spicifera and comprising the sequence as shown in SEQ ID NO:4. The potential to bleach stains was assessed by washing cotton swatches soiled with tomato stains.

The experiments were performed in small 250 ml containers, to which 15 ml of wash solution were added (indicated in tables). The pH of the wash solution was set to pH 9. Purified phenol oxidizing enzyme obtainable from Bipolaris spicifera and having an amino acid sequence as shown in SEQ ID NO:4 was added to the wash solution at a concentration of 100mg/l. Phenothiazine-10-propionate (PTP) was used as an enhancers, dosed at 250 μM. The following formulation was used as wash solution (2 gr/liter):

Detergent Composition:

| | |
|---|---|
| LAS | 24% |
| STP | 14.5% |
| Soda ash | 17.5% |
| Silicate | 8.0% |
| SCMC | 0.37% |
| Blue pigment | 0.02% |
| Moisture/salts | 34.6% |

The swatches were washed during 30 minutes, at 30° C. After the wash, the swatches were tumble-dried and the reflectance spectra were measured using a Minolta spectrometer. The color differences between the swatch before and after the wash data were expressed in the CIELAB L*a*b* color space. In this color space, L* indicates lightness and a* and b* are the chromaticity coordinates. Color differences between two swatches are expressed as DE, which is calculated from the equation:
ΔE=√ΔL²+Δa²+Δb²

The results, as ΔE values, are shown in Table 2 below:

| Wash without bleach system | Wash with bleach system |
|---|---|
| ΔE = 4.8 | ΔE = 6.9 |

As can be seen from DE values, the bleaching of the tomato stain is improved in the presence of the enzyme/enhancer system.

1-50. (canceled)

51. An isolated phenol oxidizing enzyme encoded by a nucleic acid capable of hybridizing to the nucleic acid having the sequence as shown in SEQ ID NO:1 or a fragment thereof, under conditions of high stringency which includes hybridization at about 37° C. in buffer including 50% formamide and washing at about 65° C., said isolated phenol oxidizing enzyme having at least 80% sequence identity to the amino acid sequence of SEQ ID NO: 2, wherein said isolated phenol oxidizing is obtainable from a source other than Stachybotrys chartarum.

52. The phenol oxidizing enzyme of claim 51 having at least 85% identity to the phenol oxidizing enzyme having the amino acid sequence as disclosed in SEQ ID NO:2.

53. The phenol oxidizing enzyme of claim 51, wherein said enzyme is obtained from a bacteria, yeast or non-Stachybotrys fungus.

54. The phenol oxidizing enzyme of claim 53, wherein said fungus is selected from the group consisting of Myrothecium species, Curvularia species, Chaetomium species, Bipolaris species, Humicola species, Pleurotus species, Trichoderma species, Mycellophthora species and Amerosporium species.

55. The phenol oxidizing enzyme of claim 54, wherein the fungus are selected from the group consisting of Myrothecium verrucaria, Curvalaria pallescens, Chaetomium sp, Bipolaris spicifera, Humicola insolens, Pleurotus abalonus, Trichoderma reesei, Mycellophthora thermophila and Amerosporium atrum.

56. The phenol oxidizing enzyme of claim 54, wherein said fungus is a Bipolaris species, a Curvularia species or a Amerosporium species.

57. The phenol oxidizing enzyme of claim 56, wherein said fungus is Bipolaris spicifera, Curvularia pallescens or Amerosporium atrum.

58. The phenol oxidizing enzyme of claim 51 having at least 90% sequence identity to SEQ ID NO: 4, SEQ ID NO: 7 or SEQ ID NO: 9.

59. An isolated phenol oxidizing enzyme comprising an amino acid sequence having at least 95% identity to the amino acid sequence disclosed in SEQ ID NO: 4, SEQ ID NO: 7 or SEQ ID NO: 9.

60. The isolated phenol oxidizing enzyme of claim 59 having the sequence of SEQ ID NO: 9.

61. A method for producing a phenol oxidizing enzyme in a host cell comprising the steps of: a) introducing a polynucleotide encoding the isolated phenol oxidizing enzyme according to claim 51 into a host cell and b) growing said host cell under condition suitable for the production of the phenol oxidizing enzyme.

62. The method of claim 61, wherein said polynucleotide comprises the sequence of SEQ ID NO: 6 or SEQ ID NO: 8.

63. The method of claim 61, wherein said host cell includes filamentous fungus, yeast or bacteria.

64. The method of claim 63, wherein said yeast is selected from the group consisting of Saccharomyces, Pichia, Schizosaccharomyces, Hansenula, Kluyveromyces, and Yarrowia species.

65. The method of claim 63, wherein said filamentous fungus is selected from the group consisting of Aspergillus species, Trichoderma species and Mucor species.

66. The method of claim 61 further comprising recovering the phenol oxidizing enzyme from step b).
